(19) United States
(12) Patent Application Publication
(10) Pub. No.: US 2025/0332543 A1
(43) Pub. Date: Oct. 30, 2025

(54) Switchable Dual Functional Material

(71) Applicant: University of Surrey, Guildford, Surrey (GB)

(72) Inventors: Tomas Ramirez Reina, Guildford, Surrey (GB); Loukia Pantzechroula Merkouri, Guildford, Surrey (GB); Melis Seher Duyar, Guildford, Surrey (GB)

(21) Appl. No.: 18/861,064

(22) PCT Filed: May 18, 2023
(86) PCT No.: PCT/GB2023/051311

§ 371 Date: Oct. 28, 2024

(30) Foreign Application Priority Data
May 20, 2022 (GB) 2207416.5

(51) Int. Cl. B01D 53/86; B01D 53/04; B01D 53/62; B01D 53/73; B01J 21/04; B01J 23/89; B01J 35/61; B01J 35/63; B01J 37/12; C10G 2/00

(52) U.S. Cl. CPC B01D 53/8671; B01D 53/0407; B01D 53/62; B01D 53/73; B01J 21/04; B01J 23/892; B01J 35/615; B01J 35/633; B01J 37/12; C10G 2/50; B01D 2255/20753; B01D 2257/504

(57) ABSTRACT

The disclosure provides a method of converting carbon dioxide into a reaction product. The method comprises providing a switchable dual function material (DFM) loaded with carbon dioxide; and contacting the switchable DFM loaded with carbon dioxide and a co-reactant, thereby causing the carbon dioxide to react with the co-reactant to produce the reaction product. The switchable DFM comprises an adsorbent, configured to adsorb carbon dioxide; and a switchable catalyst configured to catalyse the conversion of carbon dioxide into a reaction product. The disclosure extends to the switchable DFM per se.

The invention relates to a dual function material (DFM), and more specifically a switchable DFM which may be used to capture carbon dioxide, and to catalyse the conversion of carbon dioxide into a reaction product. The invention also extends to a method of producing the switchable DFM and methods of using the switchable DFM.

Global warming is increasing at an alarming rate, as carbon dioxide (CO₂) emissions reached 33 Gt in 2021 [1]. The CO₂ concentration in the atmosphere has gone up from approximately 270 ppm in the pre-industrial era to 420 ppm in 2022, signifying a 50% increase [1-3]. CO₂ is associated with global warming and is considered the main greenhouse gas due to the global dependency on fossil fuels for energy, transportation, and industrial purposes. Efforts are continuously made to control greenhouse gas concentrations and thus the extent of global warming, leading to the Paris Agreement in 2015. Its goal was to hold the average temperature rise below 2° C. above pre-industrial levels and to take concerted action to keep the temperature increase below 1.5° C. above pre-industrial levels in order to reduce the anthropogenic impact on climate change [4]. Nonetheless, in 2021 the global average rise was 1° C. compared to 1880 as reported by NASA [5]. Consequently, the Intergovernmental Panel on Climate Change (IPCC) stresses the need to achieve net zero CO₂ emissions by 2050 and have a substantial decrease in CO₂ emissions after 2030 [6].

A solution to control CO₂ emissions is Carbon Capture and Storage (CCS). During this process, the CO₂ is captured from several industrial effluent streams, transported, injected into the lower earth layers, and stored in geological formations or into the oceans at great depths [7]. The current state-of-the-art technologies in terms of CO₂ capture includes the widely commercialised amine absorption and monoethanolamine (MEA) in particular. Due to their corrosive nature, amines have to be diluted with water (20-30% amine in water solution). In the regeneration step, during which it is necessary to increase the temperature in order to break the amine-CO₂ bonds, the high water content leads to a significant energy consumption [8-10]. Consequently, the high energy requirements of the CCS process are translated into high capital and operating costs, which, in turn, account for its slow commercialisation. More specifically, it is estimated that 75% of the total CCS cost is attributed to the CO₂ capture and compression [11]. An alternative to amine absorption is solid adsorption, e.g. metal organic frameworks MOFs, zeolites, and alkali/alkaline oxides/carbonates. During adsorption, CO₂ is either chemisorbed or physisorbed onto the adsorbent's surface until breakthrough, or saturation, is reached. The desorption of a concentrated CO₂ stream usually occurs via a pressure or temperature swing process [8,12].

Another solution to control CO₂ emissions is Carbon Capture and Utilisation (CCU), during which the CO₂ is used to produce added-value chemicals and fuels instead of simply being sequestered. The CCU approach is considered a more active and sustainable way because CO₂ is an economically viable, safe, and renewable carbon source, which can be used as a C₁ building block for the production of fuels and chemicals. These chemicals include synthetic natural gas, synthesis gas or syngas, urea, methanol, long chain hydrocarbons via Fischer-Tropsch synthesis, among many others. It is most commonly used in the production of urea (˜160 Mt/year). However, CO₂ being a highly stable molecule thermodynamically, it needs catalysts for its conversion into various products [11,13,14].

As far as the CO₂ catalytic upgrading routes are concerned, the dry reforming of methane (DRM) [15-17], the reverse water-gas shift (RWGS) [2,18-20] and the CO₂ methanation reactions [18,21,22] have attracted a lot of attention in the recent years. In DRM, two of the most harmful greenhouse gases react to obtain syngas, which is a mixture of carbon monoxide (CO) and hydrogen (H₂). DRM can be used for biogas upgrading purposes because biogas is mainly a mixture of CO₂ and methane (CH₄). Although research efforts are concentrated on finding the best catalytic formulation for DRM, deactivation via coke formation is inevitable at high operating temperatures [11,15]. RWGS, which is one of the CO₂ hydrogenation reactions, results in the formation of CO and water. CO is an important raw material for the production of methanol and hydrocarbons via the Fischer-Tropsch synthesis. Careful catalyst design is necessary, however, due to the occurrence of the forward reaction [11,13,18]. CO₂ methanation, which is another CO₂ hydrogenation reaction, can be used for the production of CH₄, or synthetic natural gas. This reaction attracted a lot of attention both in the 1970s, during the oil crisis, and nowadays, because of its possible use in power to gas schemes. Active catalysts are needed for CO₂ reduction and for overcoming significant kinetic limitations [11,13,23]. The aforementioned reactions are presented below.

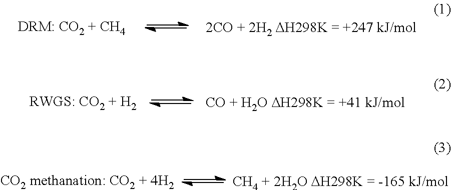

The use of Dual Function Materials (DFMs) for the integration of CO₂ capture and utilisation has been proposed recently as an alternative to the high energy demands and costs of CCU processes [24]. DFMs were introduced as a solution to the corrosive amines, the thermal swing process and the complex purification and transportation systems. They are designed to work in cyclic operation, i.e. in successive CO₂ capture and reduction cycles. They are composed of an adsorbent and a catalyst, both dispersed onto the same support. Consequently, DFMs are able to capture the CO₂ from an effluent stream and then to catalyse it to produce various chemicals based on the co-reactant used. The adsorbents used are usually alkali/alkaline earth oxides/carbonates dispersed onto alumina oxide (Al₂O₃), resulting in an increased number of adsorption sites. DFMs make use of the mid-temperature chemisorbents, which can easily be regenerated by an inert gas purge stream. As regards the catalytic component, it depends on the targeted reaction. The most studied materials are the noble metals, like ruthenium (Ru) and rhodium (Rh) due to their increased catalytic activity in the DRM, RWGS and CO₂ methanation reactions. However, their high cost is prohibitive and efforts are made to use cheaper, but highly active, materials, like nickel (Ni) [25-28]. In a typical DFM configuration, as it was initially described for the first time in [29], two reactors are needed to run in parallel, one performing the CO₂ capture step and the other the CO₂ reduction step. Therefore, the DFMs are first exposed to a stream containing CO₂ until breakthrough is reached. Then, the co-reactant is introduced to the system and the CO₂ is spilled over from the adsorbents onto the catalyst and it is converted into the desired product [30,31].

The most studied reaction for the DFMs application to date is CO₂ methanation [25-28]. This reaction offers an isothermal solution to the DFMs system because both CO₂ methanation and adsorption are exothermic processes. The exothermicity of the CO₂ methanation can supply the required heat for the CO₂ desorption and its spill-over onto the catalytic sites [24,31,32]. The development of DFMs in the RWGS and DRM is still in its infancy, but several studies in RWGS [33-37] and DRM [38-41] have shown great potential and are worthy of notice because the DFMs overcome the current limitations of cutting-edge technologies and can help in the development of carbon-negative technologies in the future.

The present invention arose in attempting to overcome the problems associated with the prior art.

In accordance with a first aspect of the invention, there is provided a switchable dual function material (DFM) comprising:
an adsorbent, configured to adsorb carbon dioxide; and
a switchable catalyst configured to catalyse the conversion of carbon dioxide into a reaction product.

Advantageously, the switchable DFM can be used to both capture carbon dioxide and convert it to an added-value chemical. This increases efficiency and lowers costs as carbon capture and conversion can be conducted in the same reactor. The inventors have also found that the presence of the catalyst synergistically improves the ability of the switchable DFM to capture carbon dioxide. Accordingly, the switchable DFM of the present invention can capture carbon dioxide from a plant exhaust and, surprisingly, can also passively capture carbon dioxide from atmospheric air. The claimed switchable DFM can therefore be used to provide carbon neutral or carbon negative processes, which are highly desirable.

In some embodiments, the adsorbent defines a support, and the switchable catalyst is disposed on the support. In an alternative embodiment, the switchable DFM comprises a separate support, and the adsorbent and the switchable catalyst are both disposed on the support. It may be appreciated that in this embodiment, the support is separate to the adsorbent.

The switchable catalyst is preferably dispersed across the support.

The switchable catalyst disposed, and preferably dispersed, on the support may be configured to catalyse the conversion of carbon dioxide in the presence of a co-reactant.

The switchable catalyst may be configured to conduct the reduction of CO₂.

The reduction of CO₂ may be conducted with a hydrocarbon as a co-reactant. In some embodiments, other co-reactants may be present. The other co-reactants may be water and/or oxygen. Accordingly, the switchable catalyst may be configured to conduct a dry reforming reaction, a bi-reforming reaction and/or a tri-reforming reaction. In some embodiments, the dry reforming reaction is a dry reforming of methane (DRM) reaction. Similarly, the bi-reforming reaction and/or a tri-reforming reaction may be a bi-reforming and/or tri-reforming reaction of methane. Alternatively, the dry reforming reaction, bi-reforming reaction and/or tri-reforming reaction may be a dry reforming reaction, bi-reforming reaction and/or tri-reforming reaction of another hydrocarbon. Accordingly, the co-reactant may be or comprise a hydrocarbon. The hydrocarbon may be a C₁₋₁₂ hydrocarbon, more preferably a C₁₋₆ hydrocarbon or a C₁₋₃ hydrocarbon. The hydrocarbon may be methane, ethane or propane. In some embodiments, the hydrocarbon is methane. The reaction product may be or comprise carbon monoxide and hydrogen.

Alternatively, or additionally, the switchable catalyst may be configured to conduct a reverse water-gas shift (RWGS) reaction. Accordingly, the co-reactant may be or comprise hydrogen. The reaction product may be or comprise carbon monoxide and water.

The switchable catalyst may be configured to conduct a CO₂ methanation reaction. Accordingly, the co-reactant may be or comprise hydrogen. The reaction product may be or comprise methane and water.

The switchable catalyst may be configured to conduct oxidative dehydrogenation of a hydrocarbon. It may be appreciated that in this reaction the CO₂ could used as a soft oxidant. The co-reactant may be or comprise a hydrocarbon. The hydrocarbon may be a C₂₋₁₂ hydrocarbon, a C₂₋₆ hydrocarbon or a C₂₋₃ hydrocarbon. The hydrocarbon may be ethane or propane. The reaction product may be or comprise carbon monoxide, an alkene and water. The alkene may have a corresponding number of carbons as the hydrocarbon used as the co-reactant. Accordingly, the alkene may be a C₂₋₁₂ alkene, a C₂₋₆ hydrocarbon or a C₂₋₃ hydrocarbon. The alkene may be ethene or propene.

The switchable catalyst may be configured to conduct a hydrogenation reaction of the CO₂. The co-reactant may be H₂. The reaction product may be methanol and water.

A switchable catalyst may be understood to be a catalyst which can catalyse two or more different chemical reactions, wherein each reaction converts carbon dioxide into a product. The two or more different chemical reactions may be selected by varying the co-reactant and/or a reaction temperature. The two or more different chemical reactions may be two or more reactions selected from the group consisting of a dry reforming reaction; a CO₂ methanation; an RWGS reaction; a bi-reforming reaction; a tri-reforming reaction; a dehydrogenation reaction; and a hydrogenation reaction. More preferably, the two or more different chemical reactions may be two or more reactions selected from the group consisting of a dry reforming reaction; a CO₂ methanation; and an RWGS reaction. Preferably, the catalyst is able to switch between the different chemical reactions on demand.

In a preferred embodiment, the switchable catalyst is configured to be able to catalyse a dry reforming reaction and a CO₂ methanation. The switchable catalyst is preferably also configured to be able to catalyse an RWGS reaction.

The inventors have surprisingly found that the switchable DFMs described herein can switch between reactions by varying the co-reactant and/or temperature, and maintain 100% selectivity. The inventors have also surprisingly found that despite the large variation in operating temperatures for the different reactions, the adsorbents in the DFMs are capable of functioning at the full range of temperatures over which the catalyst may be used. For instance, the DFM may be used to adsorb carbon dioxide and to catalyse the conversion of carbon dioxide into a reaction product at temperatures up to 1,000° C. For instance, the DFM may be used to adsorb carbon dioxide and to catalyse the conversion of carbon dioxide into a reaction product at temperatures between 100 and 1,000° C., between 25° and 750° C. or between 35° and 650° C.

Advantageously, the same DFM can be used for two or three different reactions. This eliminates the need to have three different process and/or change materials when the process changes. Accordingly, the switchable DFM can be used in a more cost-effective manner than prior art catalysts and adsorbents. Furthermore, since the reaction may be varied if demand for the products changes, this ensures any plants incorporating the switchable DFMs of the invention are future proofed.

The switchable catalyst may comprise one or more metals and/or a metal phosphide. The one or more metals may be or comprise one or more transition metals. The metal phosphide may comprise a transition metal phosphide.

Accordingly, the switchable catalyst may comprise one or more of nickel (Ni), ruthenium (Ru), cerium (Ce), zirconium (Zr), iron (Fe), and/or an oxide or phosphide thereof.

The catalyst preferably comprises nickel. The switchable DFM may comprise at least 1 wt %, at least 2 wt %, at least 4 wt %, at least 6 wt %, at least 8 wt %, at least 10 wt %, at least 12 wt %, at least 14 wt % or at least 15 wt % nickel. The switchable DFM may comprise less than 60 wt %, less than 50 wt %, less than 40 wt %, less than 30 wt %, less than 25 wt %, less than 20 wt %, less than 18 wt %, or less than 16 wt % nickel. The switchable DFM may comprise between 2 and 60 wt %, between 4 and 50 wt %, between 6 and 40 wt %, between 8 and 30 wt %, between 10 and 25 wt %, between 12 and 20 wt %, between 13 and 18 wt % or between 14 and 16 wt % nickel. The switchable DFM may comprise about 15 wt % nickel.

In some embodiments, the catalyst consists of nickel. In alternative embodiments, the switchable catalyst comprises nickel and one or more additional components or promoters. The or each additional component or promoter may be a metal, and is preferably a transition metal. The transition metal may be ruthenium (Ru), cerium (Ce), zirconium (Zr) and/or iron (Fe).

In some embodiments, the switchable catalyst comprises an additional component or promoter, and the additional component or promoter is ruthenium. Accordingly, the switchable catalyst may comprise or consist of nickel (Ni) and ruthenium (Ru), more preferably a nickel ruthenium alloy.

Alternatively, or additionally, switchable catalyst comprises an additional component or promoter, and the additional component or promoter is iron. Accordingly, the switchable catalyst may comprise or consist of nickel (Ni) and iron (Fe), more preferably a nickel iron alloy.

In one embodiment, the switchable catalyst comprises two additional components or promoters. The additional components or promoters may be ruthenium and iron. Accordingly, the switchable catalyst may comprise or consist of nickel (Ni), ruthenium (Ru) and iron (Fe), more preferably a nickel, ruthenium and iron alloy.

The nickel and each additional component or promoter may be present at any weight ratio. For instance, the nickel and the or each additional component or promoter may be present in a weight ratio of between 1:1 and 100:1, between 2:1 and 75:1, between 4:1 and 50:1, between 6:1 and 40:1, between 8:1 and 30:1, between 10:1 and 20:1, between 12:1 and 18:1 or between 14:1 and 16:1. The nickel and ruthenium may be present in a weight ratio of about 15:1.

In an alternative embodiment, the switchable catalyst may comprise or consist of a nickel phosphide. Preferably, the nickel phosphide is a nickel rich nickel phosphide. Any ratio of nickel to phosphorus may be used. For instance, the molar ratio of nickel to phosphorous in the nickel phosphide may be between 1:1 and 5:1, between 2:1 and 3:1 or between 11:5 and 13:5. The molar ratio of nickel to phosphorous in the nickel phosphide may be about 12:5. Accordingly, the nickel phosphide may be Ni₁₂P₅. Synthesis of a suitable catalyst is described in Zhang et al. (“Ni-Phosphide catalysts as versatile systems for gas-phase CO₂ conversion: Impact of the support and evidences of structure-sensitivity”, Fuel, 323 (2022) 124301).

The switchable catalyst may comprise at least 1 wt %, at least 2 wt %, at least 4 wt %, at least 6 wt %, at least 8 wt %, at least 10 wt %, at least 12 wt %, at least 14 wt % or at least 15 wt % of the switchable DFM. The switchable catalyst may comprise less than 60 wt %, less than 50 wt %, less than 40 wt %, less than 30 wt %, less than 25 wt %, less than 20 wt %, less than 18 wt %, or less than 17 wt % of the switchable DFM. The switchable catalyst may comprise between 2 and 60 wt % of the switchable DFM, between 4 and 50 wt % of the switchable DFM, between 6 and 40 wt % of the switchable DFM, between 8 and 30 wt % of the switchable DFM, between 10 and 25 wt % switchable DFM, between 12 and 20 wt % of the switchable DFM, between 14 and 18 wt % of the switchable DFM or between 15 and 17 wt % of the switchable DFM. The switchable catalyst may comprise about 16 wt % of the switchable DFM.

The adsorbent is preferably dispersed across the support.

The adsorbent may be or comprise a metal, an oxide of a metal, a carbonate of a metal, a metal organic framework, a zeolite, silica and/or carbon. The metal, the oxide of the metal or the carbonate of the metal may be an alkali metal, an alkaline earth metal, a transition metal, a p-block metal, a lanthanide or an oxide or carbonate thereof. In some embodiments, the adsorbent may be or comprise an alkali metal, an alkaline earth metal and/or an oxide or carbonate thereof. Accordingly, the adsorbent may be or comprise sodium oxide, potassium oxide and/or calcium oxide. In one preferred embodiment, the adsorbent comprises or consists of calcium oxide.

The adsorbent may comprise at least 1 wt %, at least 2 wt %, at least 4 wt %, at least 6 wt %, at least 8 wt %, at least 9 wt % or at least 10 wt % of the switchable DFM. In some embodiments, the adsorbent may comprise at least 20 wt %, at least 30 wt %, at least 40 wt %, at least 50 wt %, at least 60 wt %, at least 70 wt % or at least 80 wt % of the switchable DFM. Alternatively, the adsorbent may comprise less than 40 wt %, less than 30 wt %, less than 20 wt %, less than 15 wt %, less than 12 wt % or less than 11 wt % of the switchable DFM. Particularly in embodiments where the DFM comprises a separate support and adsorbent, the adsorbent may comprise less than 40 wt % of the DFM. The adsorbent may comprise between 2 and 40 wt % of the switchable DFM, between 4 and 30 wt % of the switchable DFM, between 6 and 20 wt % of the switchable DFM, between 8 and 15 wt % of the switchable DFM or between 9 and 11 wt % of the switchable DFM. The adsorbent may comprise about 10 wt % of the switchable DFM.

The support may comprise or consist of a metal, a metal oxide, silica, a metal organic framework, a zeolite or a structured carbon. The metal, metal oxide or metal organic framework may be a transition metal, a transition metal oxide, a transition metal organic framework, a p-block metal, a p-block metal oxide or a p-block metal organic framework. The support may comprise or consist of cerium, aluminium, zirconium, titanium, silicon and/or an oxide thereof. Accordingly, the support may comprise or consist of cerium (IV) oxide (CeO₂), aluminium oxide (Al₂O₃), zirconium dioxide (ZrO₂), titanium dioxide (TiO₂), and/or silica (SiO₂).

In some embodiments, the support comprises or consists CeO₂ and Al₂O₃. The support may comprise between 1 and 50 wt % CeO₂, between 5 and 40 wt % CeO₂, between 10 and 35 wt % CeO₂, between 15 and 30 wt % CeO₂, between 18 and 25 wt % CeO₂ or between 19 and 21 wt % CeO₂. The support may comprise between 50 and 99 wt % Al₂O₃, between 60 and 95 wt % Al₂O₃, between 70 and 90 wt % Al₂O₃ or between 75 and 85 wt % Al₂O₃.

Accordingly, the switchable catalyst may comprise or consist of Ni, a combination of Ni and Ru, a combination of Ni and Fe or a combination of Ni, Ru and Fe and the support may be or comprise CeO₂ and Al₂O₃.

In alternative embodiments, the support comprises or consists CeO₂ and ZrO₂. The support may comprise between 10 and 95 wt % CeO₂, between 20 and 90 wt % CeO₂, between 30 and 80 wt % CeO₂, between 40 and 70 wt % CeO₂, between 50 and 65 wt % CeO₂ or between 55 and 60 wt % CeO₂. The support may comprise between 5 and 90 wt % ZrO₂, between 10 and 80 wt % ZrO₂, between 20 and 70 wt % ZrO₂, between 30 and 60 wt % ZrO₂, between 35 and 50 wt % ZrO₂ or between 40 and 45 wt % ZrO₂.

Accordingly, the switchable catalyst may comprise or consist of Ni, a combination of Ni and Ru, a combination of Ni and Fe or a combination of Ni, Ru and Fe and the support may be or comprise CeO₂ and ZrO₂.

The support may enhance the activity of the switchable catalyst and/or may also act as a catalyst.

The support may comprise at least 5 wt %, at least 10 wt %, at least 20 wt %, at least 40 wt %, at least 50 wt %, at least 60 wt %, at least 70 wt % or at least 73 wt % of the switchable DFM. The support may comprise less than 95 wt %, less than 90 wt %, less than 85 wt %, less than 80 wt % or less than 75 wt % of the switchable DFM. The support may comprise between 40 and 95 wt % of the switchable DFM, between 50 and 90 wt % of the switchable DFM, between 60 and 85 wt % of the switchable DFM, between 70 and 80 wt % of the switchable DFM or between 73 and 75 wt % of the switchable DFM. The adsorbent may comprise about 74 wt % of the DFM.

The switchable DFM preferably has a surface area of at least 100 m²/g, at least 120 m²/g, at least 130 m²/g, at least 140 m²/g, at least 150 m²/g, at least 160 m²/g, at least 170 m²/g, at least 180 m²/g or at least 190 m²/g. The switchable DFM may have a surface area between 100 and 400 m²/g, between 120 and 350 m²/g, between 140 and 300 m²/g, between 160 and 250 m²/g, between 180 and 220 m²/g or between 190 and 200 m²/g. The surface area may be calculated using the Brunauer-Emmett-Teller (BET) equation, as described in the examples.

The switchable DFM is preferably porous. More preferably, the switchable DFM is a porous nanostructured material. The switchable DFM preferably has a pore volume of at least 0.2 cm³/g, at least 0.25 cm³/g, at least 0.3 cm³/g, at least 0.35 cm³/g or at least 0.4 cm³/g. The switchable DFM preferably has a pore volume of between 0.1 and 1.0 cm³/g, between 0.2 and 0.6 cm³/g, between 0.25 and 0.55 cm³/g, between 0.3 and 0.5 cm³/g, between 0.35 and 0.45 cm³/g or between 0.4 and 0.42 cm³/g. The pore volume may be calculated using the Barett-Joyner-Halena (BJH) method.

In accordance with a second aspect of the invention, there is provided a method of producing a switchable DFM, the method comprising:
providing a support, wherein either the support is an adsorbent, and is configured to adsorb carbon dioxide, or the method comprises disposing an adsorbent on the support, wherein the adsorbent is configured to adsorb carbon dioxide; and
disposing a switchable catalyst on the support, wherein the switchable catalyst is configured to catalyse the conversion of carbon dioxide into a reaction product;
and thereby producing a switchable DFM.

The switchable DFM may be a switchable DFM of the first aspect. Accordingly, the support, adsorbent and/or switchable catalyst may be as defined in relation to the first aspect.

In some embodiments, the method comprises disposing the adsorbent on the support.

The method may comprise disposing the adsorbent on the support prior to, at the same time or after disposing the switchable catalyst on the support. Preferably, the method comprises disposing the adsorbent on the support prior to disposing the switchable catalyst on the support.

Disposing the adsorbent on the support may comprise contacting the support with the adsorbent or an adsorbent precursor. Preferably, contacting the support with the adsorbent or an adsorbent precursor comprises contacting the support with a solution or suspension comprising a solvent and the adsorbent or adsorbent precursor, to provide a further suspension. The method may comprise subsequently drying the further suspension to remove the solvent therefrom and provide a support with the adsorbent or adsorbent precursor disposed thereon. The further suspension may be dried at an elevated temperature. The temperature may be at least 30° C., at least 40° C., at least 60° C., at least 80° C., at least 100° C. or at least 110° C. The temperature may be between 3° and 300° C., between 4° and 250° C., between 60 and 200° C., between 8° and 175° C., between 10° and 150° C. or between 110 and 130° C. The further suspension may be dried for at least 1 hour, at least 2 hours, at least 4 hours, at least 6 hours, at least 8 hours, at least 10 hours or at least 12 hours.

It may be appreciated that in embodiments where the method comprises disposing the adsorbent on the support after disposing the switchable catalyst on the support then contacting the support with the adsorbent or adsorbent precursor may comprise contacting the support and switchable catalyst with the adsorbent or adsorbent precursor.

The solvent may be water.

In some embodiments, the method comprises contacting the support with a solution or suspension comprising the solvent and the adsorbent. The adsorbent may be provided as a plurality of nanoparticles. Accordingly, the method may comprise contacting the support with a colloidal suspension comprising the solvent and the plurality of nanoparticles.

The adsorbent precursor may be a nitrate of an alkali metal, a nitrate of an alkaline earth metal, a carbonate of an alkali metal, a carbonate of an alkaline earth metal, a halide of an alkali metal, a halide of an alkaline earth metal, an acetate of an alkali metal or an acetate of an alkaline earth metal. The halide may be a chloride. Accordingly, the adsorbent precursor may be or comprise sodium nitrate (NaNO₃), potassium nitrate (KNO₃), calcium nitrate (Ca(NO₃)₂), sodium carbonate (Na₂CO₃), potassium carbonate (K₂CO₃), calcium carbonate (CaCO₃), sodium chloride (NaCl), potassium chloride (KCl) and/or calcium chloride (CaCl₂)).

In embodiments which comprise contacting the support with an adsorbent precursor, the method may comprise calcining the adsorbent precursor to provide the adsorbent. The method may comprise contacting the support with the adsorbent precursor, and subsequently calcining the adsorbent precursor to provide the adsorbent. Calcining the adsorbent precursor may comprise heating the support with the adsorbent precursor disposed thereon to an elevated temperature. The elevated temperature may be at least 100° C., at least 150° C., at least 200° C., at least 250° C., at least 300° C., at least 325° C., at least 350° C. or at least 375° C. The elevated temperature may be between 100 and 1,000° C., between 150 and 800° C., between 200 and 700° C., between 25° and 600° C., between 30° and 500° C., between 325 and 475° C., between 35° and 450° C. or between 375 and 425° C. The support with the adsorbent precursor disposed thereon may be heated to the elevated temperature for at least 30 minutes, at least 1 hour, at least 2 hours, at least 3 hours or at least 4 hours. The support with the adsorbent precursor disposed thereon may be heated to the elevated temperature for between 15 minutes and 48 hours, between 30 minutes and 24 hours, between 1 and 12 hours, between 2 and 6 hours, between 3 and 5 hours or between 3.5 and 4.5 hours.

In embodiments where the method comprises drying the support, the method may comprise calcining the adsorbent precursor after drying the support.

The amount of the adsorbent precursor used may be sufficient to provide the resultant adsorbent at an amount as defined in the first aspect.

Disposing the switchable catalyst on the support may comprise contacting the support with the switchable catalyst or a switchable catalyst precursor. Preferably, contacting the support with the switchable catalyst or switchable catalyst precursor comprises contacting the support with a solution or suspension comprising a solvent and the switchable catalyst or switchable catalyst precursor, to thereby provide a further suspension. The method may comprise subsequently drying the further suspension to remove the solvent therefrom and provide a support with the switchable catalyst or switchable catalyst precursor disposed thereon. The further suspension may be dried at an elevated temperature. The temperature may be at least 30° C., at least 40° C., at least 60° C., at least 80° C., at least 100° C. or at least 110° C. The temperature may be between 3° and 300° C., between 4° and 250° C., between 6° and 200° C., between 8° and 175° C., between 10° and 150° C. or between 11° and 130° C. The further suspension may be dried for at least 1 hour, at least 2 hours, at least 4 hours, at least 6 hours, at least 8 hours, at least 10 hours or at least 12 hours.

It may be appreciated that in embodiments where the method comprises disposing the switchable catalyst on the support after disposing the adsorbent on the support then contacting the support with the switchable catalyst or switchable catalyst precursor may comprise contacting the support and absorbent with the switchable catalyst or switchable catalyst precursor.

The solvent may be water.

In some embodiments, the method comprises contacting the support with a solution or suspension comprising the solvent and the switchable catalyst. The switchable catalyst may be provided as a plurality of nanoparticles. Accordingly, the method may comprise contacting the support with a colloidal suspension comprising the solvent and the plurality of nanoparticles.

The switchable catalyst precursor may be a transition metal nitrate, a transition metal nitrosyltrinitrate, a transition metal halide, a transition metal acetate, a transition metal carbonate and/or a hydrogen phosphate. The halide may be a chloride. Accordingly, the switchable catalyst precursor may be or comprise at least one of nickel(II) nitrate (NiNO₃), ruthenium(III) nitrosyltrinitrate (Ru(NO)(NO₃)₃), iron nitrate (Fe(NO₃)₃), nickel chloride (NiCl₂), ruthenium chloride (RuCl₃), iron chloride (FeCl₃), nickel acetate, ruthenium acetate, iron acetate, nickel carbonate, ruthenium carbonate, iron carbonate and/or diammonium hydrogen phosphate [(NH₄)₂HPO₄]. In some embodiments, the switchable catalyst precursor comprises a combination of nickel(II) nitrate (NiNO₃) and ruthenium(III) nitrosyltrinitrate (Ru(NO)(NO₃)₃). In some embodiments, the switchable catalyst precursor comprises a combination of nickel(II) nitrate (NiNO₃) and iron nitrate (Fe(NO₃)₃). In some embodiments, the switchable catalyst precursor comprises a combination of nickel(II) nitrate (NiNO₃), ruthenium(III) nitrosyltrinitrate (Ru(NO)(NO₃)₃) and iron nitrate (Fe(NO₃)₃). In alternative embodiments, the switchable catalyst precursor comprises a combination of nickel(II) nitrate (NiNO₃) and diammonium hydrogen phosphate [(NH₄)₂HPO₄].

In embodiments which comprise contacting the support with a switchable catalyst precursor, the method may comprise calcining the switchable catalyst precursor to provide the switchable catalyst. The method may comprise contacting the support with the switchable catalyst precursor, and subsequently calcining the switchable catalyst precursor to provide the switchable catalyst. Calcining the switchable catalyst precursor may comprise heating the support with the switchable catalyst precursor disposed thereon to an elevated temperature. The elevated temperature may be at least 100° C., at least 150° C., at least 200° C., at least 250° C., at least 300° C., at least 350° C., at least 400° C., at least 450° C. or at least 475° C. The elevated temperature may be between 100 and 1,000° C., between 15° and 900° C., between 20° and 800° C., between 25° and 700° C., between 400 and 600° C., between 55° and 550° C. or between 475 and 525° C. The support with the switchable catalyst precursor disposed thereon may be heated to the elevated temperature for at least 30 minutes, at least 1 hour, at least 2 hours, at least 3 hours or at least 4 hours. The support with the adsorbent precursor disposed thereon may be heated to the elevated temperature for between 5 minutes and 48 hours, between 15 minutes and 24 hours, between 30 minutes and 12 hours, between 1 and 6 hours, between 2 and 4 hours or between 2.5 and 3.5 hours.

In accordance with a third aspect, there is provided use of the switchable DFM of the first aspect to capture carbon dioxide.

In accordance with a fourth aspect, there is provided use of the switchable DFM of the first aspect to convert carbon dioxide into a reaction product.

In accordance with a fifth aspect, there is provided a method of capturing carbon dioxide, the method comprising contacting a gas comprising carbon dioxide and the switchable DFM of the first aspect.

The carbon dioxide may be present in the gas at a concentration of at least 0.01 vol %, at least 0.02 vol % or at least 0.04 vol %. Accordingly, the gas comprising carbon dioxide may be atmospheric gas. Accordingly, the carbon dioxide may be present in the gas at a concentration of less than 80 vol %, less than 60 vol %, less than 40 vol %, less than 20 vol %, less than 10 vol %, less than 5 vol %, less than 1 vol %, less than 0.1 vol % or less than 0.05 vol %. The carbon dioxide may be present in the gas at a concentration of between 0.0001 and 40 vol %, between 0.0005 and 20 vol %, between 0.001 and 10 vol %, between 0.005 and 5 vol %, between 0.01 and 1 vol %, between 0.02 and 0.1 vol % or between 0.04 and 0.05 vol %.

Alternatively, the carbon dioxide may be present in the gas at a concentration of at least 0.1 vol %, at least 0.5 vol %, at least 1 vol %, at least 2 vol %, at least 4 vol %, at least 6 vol %, at least 8 vol % or at least 10 vol %. For instance, the gas may be exhaust gas produced by an industrial plant. The carbon dioxide may be present in the gas at a concentration between 0.01 and 80 vol %, between 0.02 and 70 vol %, between 0.04 and 60 vol %, between 0.1 and 50 vol %, between 0.5 and 40 vol %, between 1 and 30 vol %, between 2 and 25 vol %, between 4 and 20 vol %, between 6 and 15 vol %, between 8 and 12 vol % or between 9 and 11 vol %.

The method may comprise contacting the gas comprising carbon dioxide and the switchable DFM at room temperature. Alternatively, the method may comprise contacting the gas comprising carbon dioxide and the switchable DFM at an elevated temperature. Accordingly, the method may comprise contacting the gas comprising carbon dioxide and the switchable DFM at a temperature between 0 and 1,500° C., between 20 and 1,250° C., between 50 and 1,000° C., between 10° and 900° C., between 15° and 850° C., between 20° and 800° C., between 250 and 750° C., between 30° and 700° C. or between 35° and 650° C.

In one embodiment, the method may comprise contacting the gas comprising carbon dioxide and the switchable DFM at a temperature between 10° and 600° C., between 15° and 550° C., between 200 and 500° C., between 250 and 450° C., between 300 and 400° C. or between 325 and 375° C. In an alternative embodiment, the method may comprise contacting the gas comprising carbon dioxide and the switchable DFM at a temperature between 350 and 1,000° C., between 400 and 900° C., between 45° and 850° C., between 50° and 800° C., between 550 and 750° C., between 60° and 700° C. or between 625 and 675° C.

The method may be conducted under steady state, dynamic conditions or a combination thereof.

The method may be considered to be conducted under steady state if it is conducted at a fixed temperature. For instance, it may be appreciated that if the method is conducted at a fixed temperature then the temperature may remain substantially the same while the gas comprising carbon dioxide and the switchable DFM are contacted. The temperature may be understood to stay substantially the same if it does not vary by more than 100° C., more preferably if it does not vary by more than 50° C., if it does not vary by more than 25° C. or if it does not vary by more than 10° C.

Alternatively, the method may be considered to be conducted under dynamic conditions if the temperature varies while the gas comprising carbon dioxide and the switchable DFM are contacted. The temperature may either increase or decrease as the gas comprising carbon dioxide and the switchable DFM are contacted. Preferably, the temperature increases as the gas comprising carbon dioxide and the switchable DFM are contacted. The temperature may vary at a rate of at least 0.1° C./min, at least 0.5° C./min, at least 1° C./min, at least 2.5° C./min, at least 5° C./min, at least 7.5° C./min or at least 10° C./min. The temperature may vary at a rate of between 0.1 and 50° C./min, between 0.5 and 40° C./min, between 1 and 30° C./min, between 2.5 and 20° C./min, between 5 and 15° C./min or between 7.5 and 12.5° C./min.

When the method is conducted under dynamic conditions, the entire method may be conducted within a temperature range as defined above.

The captured carbon dioxide may be released from the switchable DFM. The captured carbon dioxide may be released by exposing the DFM to different conditions, such as purging with steam. The resulting carbon dioxide may be stored. Alternatively, the carbon dioxide may be used in industry without the need for conversion. In particular, due to having been captured by the DFM, the carbon dioxide has been purified and so could be a valuable product in its own right. For instance, the purified carbon dioxide may be used in the beverage industry.

Alternatively, the carbon dioxide may be converted into a reaction product.

In accordance with a sixth aspect, there is provided a method of converting carbon dioxide into a reaction product, the method comprising:
providing a switchable DFM, as defined in the first aspect, loaded with carbon dioxide; and
contacting the switchable DFM loaded with carbon dioxide and a co-reactant, thereby causing the carbon dioxide to react with the co-reactant to produce the reaction product.

Providing the switchable DFM loaded with carbon dioxide may comprise conducting the method of the fifth aspect. The step of contacting the switchable DFM loaded with carbon dioxide and the co-reactant may be conducted subsequent to the step of contacting a gas comprising carbon dioxide and the switchable DFM.

The method may comprise selecting a specific co-reactant and/or reaction temperature to provide a desired reaction product.

The co-reactant may be selected from the group consisting of a hydrocarbon; hydrogen; water and oxygen. The hydrocarbon may be as defined above.

It may be appreciated that when the co-reactant is a hydrocarbon, and optionally also water and/or oxygen, the switchable catalyst may be used to conduct a dry reforming reaction, a bi-reforming reaction, a tri-reforming reaction or dehydrogenation. The reaction product for the dry reforming reaction would be carbon monoxide and hydrogen. The reaction product for the dehydrogenation reaction would be carbon monoxide, an alkene and water. The alkene may be as defined above.

Alternatively, when the co-reactant is hydrogen, the switchable catalyst may be used to conduct the RWGS, CO₂ methanation or hydrogenation reactions. The reaction product for the RWGS reaction would be carbon monoxide and water. The reaction product for the CO₂ methanation reaction would be methane and water. The reaction product for the hydrogenation reaction would be methanol and water.

Accordingly, the reaction product may be selected from the group consisting of carbon monoxide; hydrogen; carbon monoxide; methane; water; an alkene; methanol; and a combination thereof. In one embodiment, the reaction product is carbon monoxide and hydrogen. In an alternative embodiment, the reaction product is carbon monoxide and water. In a further alternative embodiment, the reaction product is methane and water. In a further alternative embodiment, the reaction product is methanol and water.

The method may comprise contacting the switchable DFM loaded with carbon dioxide and a gas comprising the co-reactant. The co-reactant may be present in the gas at a concentration of at least 0.1 vol %, at least 0.5 vol %, at least 1 vol %, at least 2 vol %, at least 4 vol %, at least 6 vol %, at least 8 vol % or at least 10 vol %. The co-reactant may be present in the gas at a concentration between 0.01 and 80 vol %, between 0.02 and 70 vol %, between 0.04 and 60 vol %, between 0.1 and 50 vol %, between 0.5 and 40 vol %, between 1 and 30 vol %, between 2 and 25 vol %, between 4 and 20 vol %, between 6 and 15 vol %, between 8 and 12 vol % or between 9 and 11 vol %.

The gas may comprise or consist of the co-reactant and nitrogen. Accordingly, nitrogen may be present in the gas at a concentration between 1 and 99.9 vol %, between 10 and 99.5 vol %, between 20 and 99 vol %, between 30 and 98 vol %, between 40 and 97 vol %, between 50 and 96 vol %, between 60 and 95 vol %, between 70 and 94 vol %, between 80 and 93 vol %, between 85 and 92 vol % or between 89 and 91 vol %.

The method may comprise contacting the switchable DFM loaded with carbon dioxide and the co-reactant at an elevated temperature. Accordingly, the method may comprise contacting the gas comprising carbon dioxide and the switchable DFM at a temperature between o and 1,500° C., between 20 and 1,250° C., between 50 and 1,000° C., between 10° and 900° C., between 15° and 850° C., between 20° and 800° C., between 25° and 750° C., between 300 and 700° C., between 35° and 650° C. or between 40° and 600° C.

In one embodiment, the method may comprise contacting the switchable DFM loaded with carbon dioxide and the co-reactant at a temperature between 10° and 600° C., between 15° and 550° C., between 200 and 500° C., between 25° and 450° C., between 30° and 400° C. or between 325 and 375° C. In one embodiment, the temperature may be about 350° C.

In an alternative embodiment, the method may comprise contacting the switchable DFM loaded with carbon dioxide and the co-reactant at a temperature between 350 and 1,000° C. or between 40° and 900° C. The method may comprise contacting the switchable DFM loaded with carbon dioxide and the co-reactant at a temperature between 45° and 850° C., between 500 and 800° C., between 55° and 750° C., between 60° and 700° C. or between 625 and 675° C. The temperature may be about 650° C.

Alternatively, the method may comprise contacting the switchable DFM loaded with carbon dioxide and the co-reactant at a temperature between 40° and 800° C., between 45° and 750° C., between 50° and 700° C., between 55° and 650° C. or between 575 and 625° C. The temperature may be about 600° C.

In a further alternative embodiment, the method may comprise contacting the switchable DFM loaded with carbon dioxide and the co-reactant at a temperature between 450 and 1,000° C., between 50° and 950° C., between 60° and 900° C., between 65° and 850° C. or between 70° and 800° C.

In a still further embodiment, the method may comprise contacting the switchable DFM loaded with carbon dioxide and the co-reactant at a temperature between 75 and 600° C., between 100 and 550° C., between 125 and 500° C., between 15° and 450° C., between 175 and 350° C. or between 20° and 300° C.

The method may be conducted under steady state, dynamic conditions or a combination thereof.

The method may be considered to be conducted under steady state if it is conducted at a fixed temperature. For instance, it may be appreciated that if the method is conducted at a fixed temperature then the temperature may remain substantially the same while the switchable DFM loaded with carbon dioxide and the co-reactant are contacted. The temperature may be understood to stay substantially the same if it does not vary by more than 100° C., more preferably if it does not vary by more than 50° C., if it does not vary by more than 25° C. or if it does not vary by more than 10° C.

Alternatively, the method may be considered to be conducted under dynamic conditions if the temperature varies while the switchable DFM loaded with carbon dioxide and the co-reactant are contacted. The temperature may either increase or decrease as the switchable DFM loaded with carbon dioxide and the co-reactant are contacted. Preferably, the temperature increases as switchable DFM loaded with carbon dioxide and the co-reactant are contacted. The temperature may vary at a rate of at least 0.1° C./min, at least 0.5° C./min, at least 1° C./min, at least 2.5° C./min, at least 5° C./min, at least 7.5° C./min or at least 10° C./min. The temperature may vary at a rate of between 0.1 and 50° C./min, between 0.5 and 40° C./min, between 1 and 30° C./min, between 2.5 and 20° C./min, between 5 and 15° C./min or between 7.5 and 12.5° C./min.

When the method is conducted under dynamic conditions, the entire method may be conducted within a temperature range as defined above.

The switchable DFM loaded with carbon dioxide and the co-reactant may be contacted at atmospheric pressure. Alternatively, the switchable DFM loaded with carbon dioxide and the co-reactant may be contacted at an elevated pressure. The elevated pressure may be at least 2 bar, at least 5 bar, at least 10 bar, at least 20 bar, at least 30 bar, at least 40 bar or at least 50 bar. The switchable DFM loaded with carbon dioxide and the co-reactant may be contacted at a pressure between 0.01 and 1,000 bar, between 0.1 and 100 bar or between 0.5 and 55 bar. In one embodiment, the switchable DFM loaded with carbon dioxide and the co-reactant may be contacted at a pressure between 0.5 and 40 bar, between 0.6 and 20 bar, between 0.7 and 10 bar, between 0.8 and 5 bar or between 0.9 and 2 bar. In an alternative embodiment, the switchable DFM loaded with carbon dioxide and the co-reactant may be contacted at a pressure between 10 and 90 bar, between 20 and 80 bar, between 30 and 70 bar, between 40 and 60 bar or between 45 and 55 bar.

The inventors have found that by selecting the co-reactant and the reaction temperature it is possible to maintain 100% selectivity of the reaction which is carried out, and the reaction product which is produced. In particular, when the reaction is carried out at 350° C., and the co-reactant is hydrogen, the inventors have found that the CO₂ methanation reaction is conducted. Conversely, when the reaction is carried out at 650° C., and the co-reactant is hydrogen, the inventors have found that the RWGS reaction is conducted. When the reaction is carried out between 40° and 650° C., for instance at 650° C., and the co-reactant is a hydrocarbon, the inventors have found that the dry reforming reaction is conducted. The inventors note that if the reaction were conducted between 70° and 800° C., and the co-reactant is a hydrocarbon and water then the bi-reforming reaction would take place. Similarly, if the reaction were conducted between 70° and 800° C., and the co-reactant is a hydrocarbon, water and oxygen then the tri-reforming reaction would take place. If the reaction were conducted at about 600° C. and the co-reactant was a hydrocarbon, then the dehydrogenation reaction would take place. If the reaction were conducted between 200-300° C., optionally at an elevated pressure, and the co-reactant were hydrogen, then the hydrogenation reaction would take place. As explained above, since the reaction which is conducted may be varied if demand for the products changes, this ensures any plants incorporating the DFMs of the invention are future proofed.

The method may be repeated. Accordingly, the method may comprise:
contacting a gas comprising carbon dioxide and the switchable DFM of the first aspect;
subsequently contacting the switchable DFM loaded with carbon dioxide and a co-reactant, thereby causing the carbon dioxide to react with the co-reactant to produce the reaction product; and
subsequently repeating the above two steps at least once.

Accordingly, the step of contacting a gas comprising carbon dioxide and the switchable DFM and the step of contacting the switchable DFM loaded with carbon dioxide and the co-reactant may be alternated. It may be understood that the method may comprise alternating streams of reactants (i.e. the gas comprising carbon dioxide and the co-reactant). The method could be repeated, by alternating the streams of reactants, multiple times. Accordingly, the method could be conducted continuously for at least a day, a week, a month or a year.

It is noted that deactivation of a catalyst is a problem suffered by the prior art. Prior art steady state systems are not designed to cope with alternating streams of gases. Instead, these catalysts tend to have a reactor feed comprising carbon dioxide and a co-reactant, delivered together. In these prior art systems, if a catalyst deactivates then the steady state operation must be interrupted to reactivate the catalyst. This is not desirable because it is expensive to shut down/stop/divert/delay the process.

However, due to the fact that the present method is designed to cope with alternating streams of gases, a catalyst can advantageously be reactivated without the need to shut down the entire system.

Accordingly, the method may comprise:
contacting the DFM with a reactivating gas stream, and thereby regenerating and/or reactivating the catalyst.

The reactivating gas stream may be or comprise hydrogen and/or water. It may be appreciated that water will be in the form of steam.

The above step may be conducted at the pressure and/or temperature ranges described above.

The method may comprise sensing the activity of the DFM. Sensing the activity of the DFM may comprise sensing the concentration of the reaction product in an output gas stream. The above regenerating and/or reactivating step may be conducted when the activity of the DFM falls below a predetermined activity. For instance, the above regenerating and/or reactivating step may be conducted when the concentration of the reaction product in an output gas stream falls below a predetermined concentration.

Subsequent to conducting the regenerating and/or reactivating step, the repeating steps of contacting the DFM with carbon dioxide and subsequently contacting the DFM with the co-reactant may be resumed.

All features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying drawings, in which:

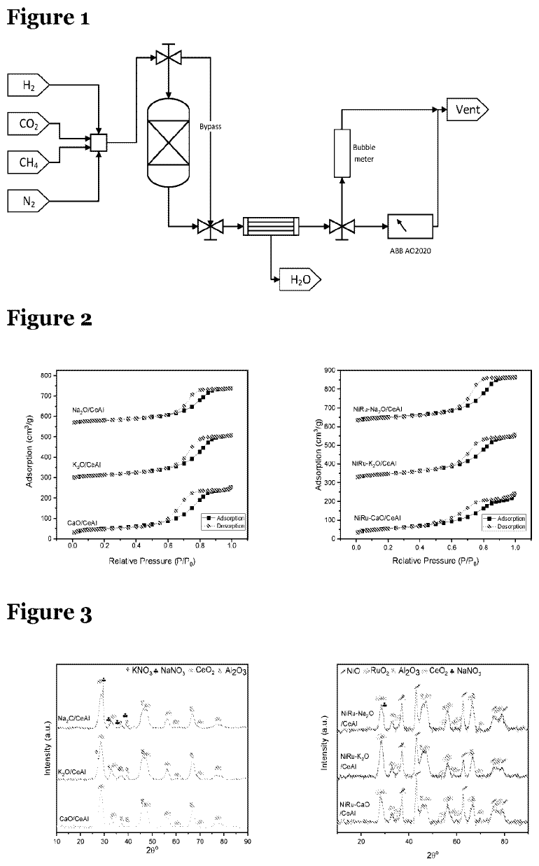

FIG. 1 is a simplified process flow diagram for experimental set up;

FIG. 2 shows nitrogen adsorption-desorption isotherms of supported adsorbents on the left-hand side and of dual functional materials (DFMs) on the right-hand side;

FIG. 3 shows X-ray diffraction (XRD) patterns of fresh supported adsorbents on the left-hand side and of DFMs on the right-hand side;

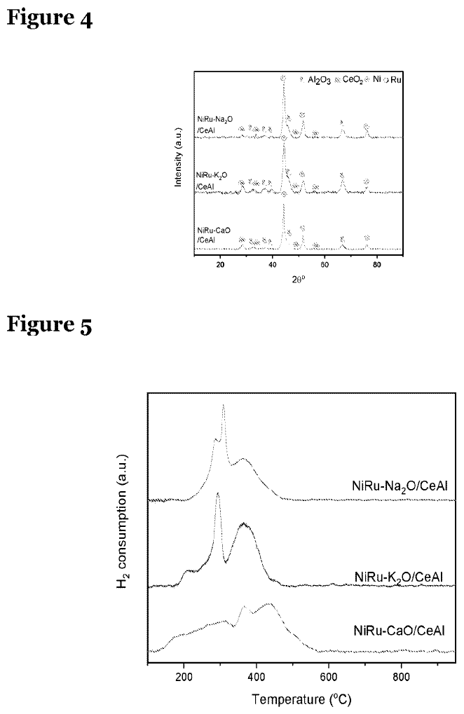

FIG. 4 shows XRD patterns of reduced DFMs;

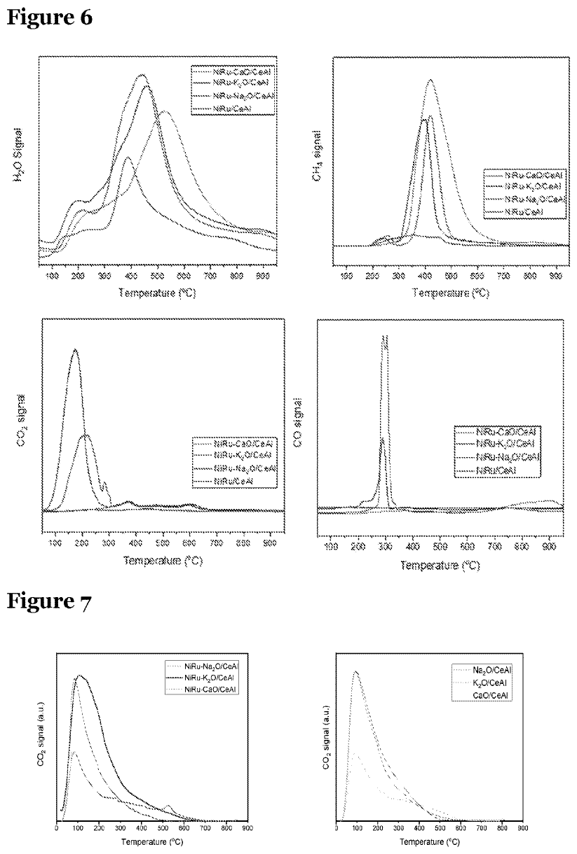

FIG. 5 shows the H₂-TPR profiles of NRNa, NRK and NRCa FIG. 6 shows the CH₄, H₂O, CO and CO₂ signals (m/z=15, 18, 28, 44) during H₂-TPR for NRNa, NRK, NRCa and NR;

FIG. 7 shows CO₂-TPD profiles of NRNa, NRK and NRCa on the left-hand side and of Na, K and Ca on the right-hand side;

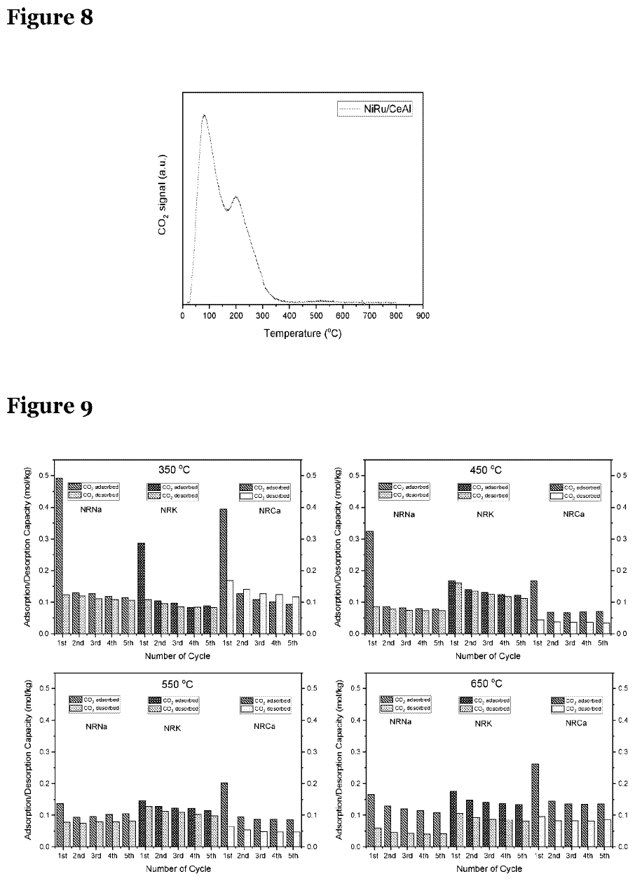

FIG. 8 shows CO₂-TPD profile of NR;

FIG. 9 shows CO₂ Adsorption/desorption cycles of NRNa, NRK and NRCa at 350, 450, 550 and 650° C.′

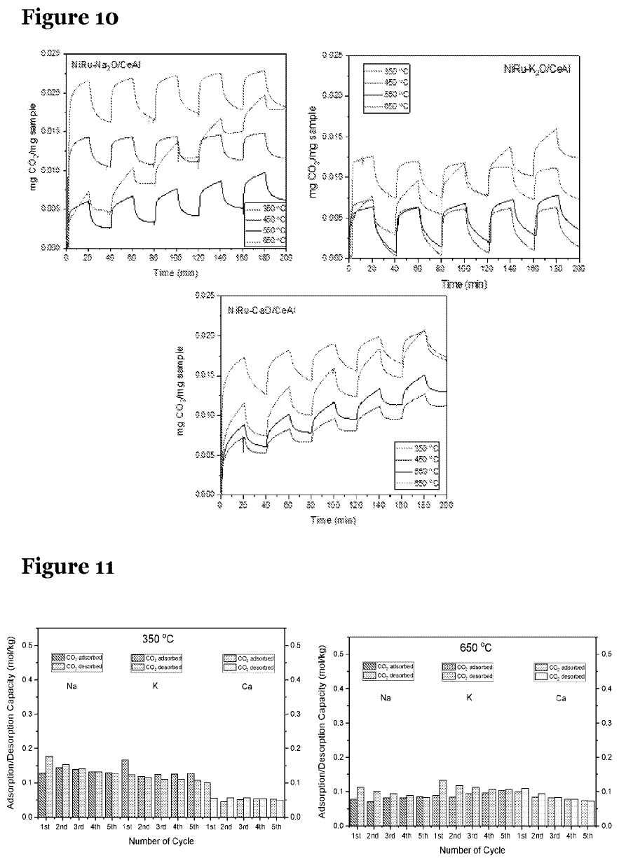

FIG. 10 shows TGA adsorption-desorption results at different temperatures for NRNa, NRK and NRCa;

FIG. 11 shows CO₂ Adsorption/desorption cycles of Na, K and Ca at 350 and 650° C.;

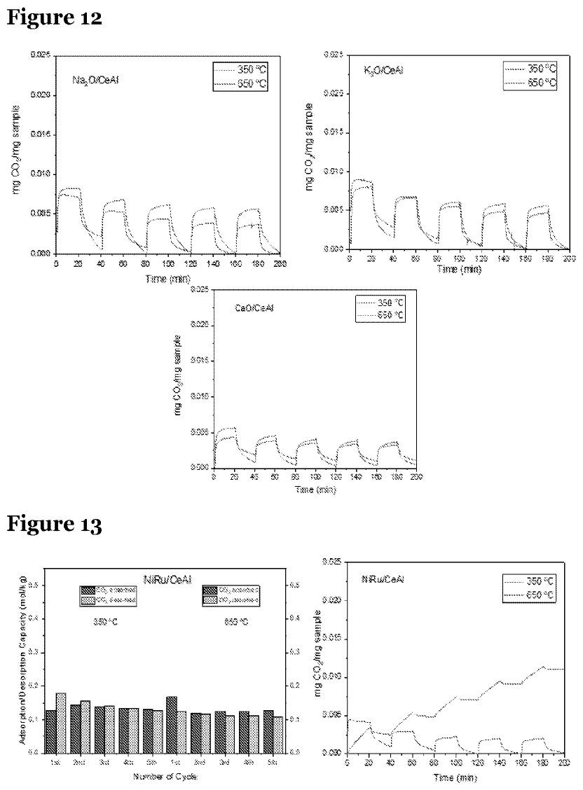

FIG. 12 shows TGA adsorption-desorption results at different temperatures for Na, K and Ca;

FIG. 13 shows TGA adsorption-desorption results at different temperatures for NR;

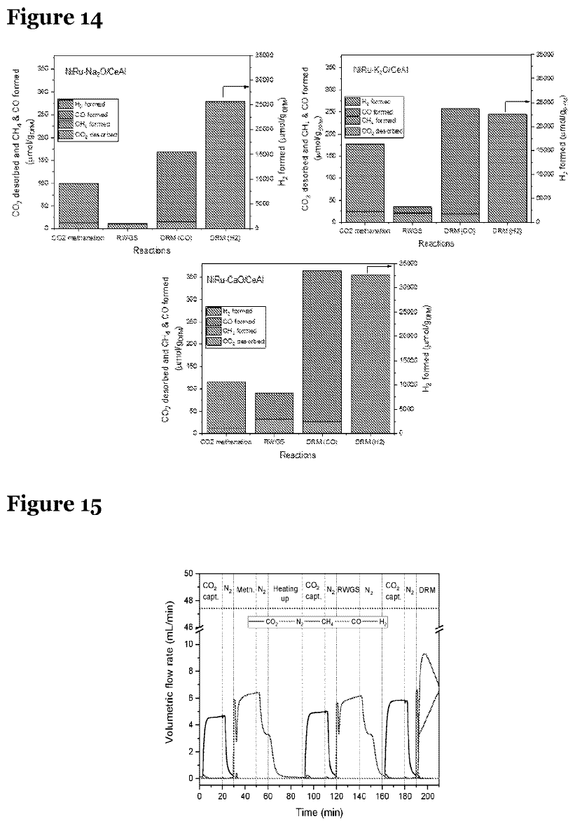

FIG. 14 shows the results of feasibility study of flexible DFMs showing the amount of CO₂ desorbed and products formation in each reaction for NRNa, NRK and NRCa;

FIG. 15 shows the CO₂ capture and CO₂ methanation, followed by the RWGS and DRM for NRNa;

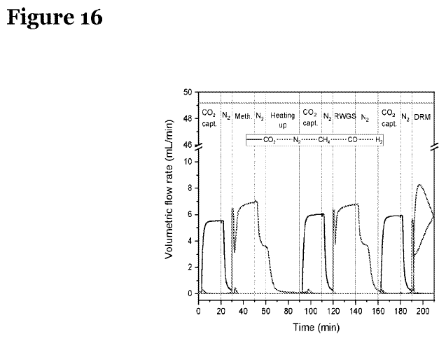

FIG. 16 shows the CO₂ capture and CO₂ methanation, followed by the RWGS and DRM for NRK;

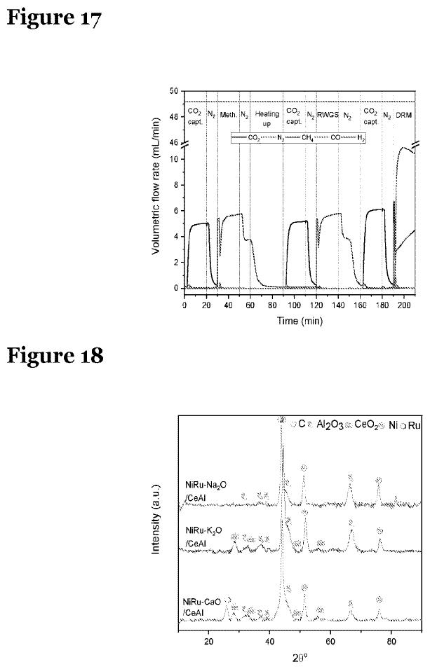

FIG. 17 shows the CO₂ capture and CO₂ methanation, followed by the RWGS and DRM for NRCa;

FIG. 18 shows XRD for post reaction NRNa, NRK and NRCa;

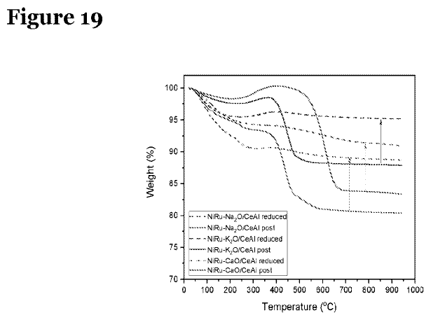

FIG. 19 shows TGA for post reaction NRNa, NRK and NRCa; and

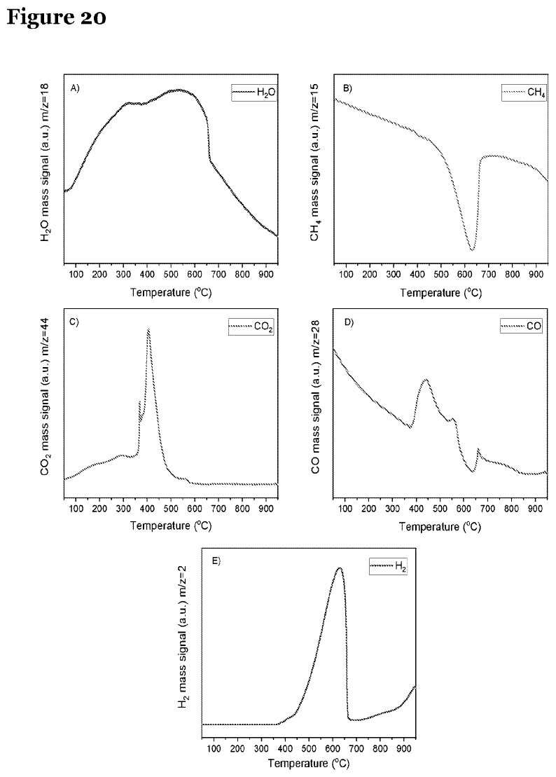

FIG. 20 shows CH₄-Temperature Programmed Surface Reaction (CH₄-TPSR) profile of NiRuCa: A) H₂O, B) CH₄, C) CO₂, D) CO and E) H₂ signals.

### EXAMPLE 1-DUAL FUNCTION MATERIALS SYNTHESIS

The three DFMs described herein were prepared by sequential impregnation. The adsorbents were impregnated onto the CeO₂—Al₂O₃ support and then, the two transition metals, Ni and Ru, were impregnated onto the supported adsorbents, as the inventors have found that this order of impregnation resulted in a better DFM performance. Therefore, the three supported adsorbents were prepared by initially mixing the required amounts of the CeO₂—Al₂O₃ support (SCFa-160 Ce20 Puralox, Sasol) and NaNO₃ (Fluka), KNO₃ (Sigma Aldrich), and Ca(NO₃)₂·4H₂O (Sigma Aldrich) with distilled water. The suspensions were then mixed at room temperature with a magnetic stirrer and the excess water was removed in a rotary evaporator under reduced pressure. Afterwards, they were dried overnight at 120° C. and calcined at 400° C. for 4 hours (5° C./min). The amount of the adsorbents' precursors used was such that the final DFMs would have 10 wt. % of their respective oxides, and thus the resulting supported adsorbents were:
Na₂O/CeO₂—Al₂O₃ (identified hereinafter as “Na”),
K₂O/CeO₂—Al₂O₃ (identified hereinafter as “K”), and
CaO/CeO₂—Al₂O₃(identified hereinafter as “Ca”).

The supported adsorbents had 11.9 wt. % Na₂O, K₂O, and CaO, respectively.

A similar impregnation procedure was performed when synthesising the three DFMs. The required amounts of Ni(NO₃)₂·6H₂O (Acros Organics) and Ru(NO)(NO₃)₃ solution (1.5 w/v Ru, Alfa Aesar) were mixed with the supported adsorbents in order to obtain 15 wt. % Ni and 1 wt. % Ru. These were mixed with excess distilled water, which was then removed in a rotary evaporator under reduced pressure, dried overnight at 120° C., and calcined at 500° C. for 3 hours (5° C./min). As a result, the prepared DFMs were:
15 wt. % Ni, 1 wt. % Ru-10 wt. % Na₂O/CeO₂—Al₂O₃(identified hereinafter as “NRNa”),
15 wt. % Ni, 1 wt. % Ru-10 wt. % K₂O/CeO₂—Al₂O₃ (identified hereinafter as “NRK”), and
15 wt. % Ni, 1 wt. % Ru-10 wt. % CaO/CeO₂—Al₂O₃ (identified hereinafter as “NRCa”).

In some experiments, the 15 wt. % Ni, 1 wt. % Ru/CeO₂—Al₂O₃ (identified hereinafter as “NR”), whose synthesis method was described in [42], was used as a reference material so as to show the impact of the addition of the adsorbents.

### Example 2—Textural Properties

The textural properties of the supported adsorbents and DFMs are presented in Table 1, below.

TABLE 1
Textural properties of the supported adsorbents and DFMs

| Sample | S_{BET} (m²/g) | V_{PORE} (cm³/g) | D_{PORE} (nm) |
|---|---|---|---|
| Na | 109 | 0.29 | 10.5 |
| K | 136 | 0.36 | 10.4 |
| Ca | 170 | 0.39 | 8.7 |
| NRNa | 173 | 0.41 | 9.4 |
| NRK | 170 | 0.40 | 8.9 |
| NRCa | 194 | 0.37 | 6.9 |

It appears that the addition of Ni and Ru during impregnation did not cover the supported adsorbents and that high specific surface area materials were formed instead. FIG. 2 shows the N₂ adsorption-desorption isotherms of the materials. The isotherms generated corresponded to the type IV isotherms with a characteristic H1 hysteresis loop, according to IUPAC classification. This type of isotherms is linked to well-developed cylindrical mesoporous materials and the high steepness was indicative of the mesopores being homogeneously distributed throughout the structure of the samples, as observed in SEM as well [43,44].

### Example 3—Crystalline Structure

FIG. 3 shows the crystalline phases of the fresh materials. All the supported adsorbents and DFMs had the characteristic peaks of γ-Al₂O₃ and CeO₂ phases (JCPDS 00-004-0880 and JCPDS 03-065-5923, respectively). No peaks corresponding to any species of adsorbents appeared in the XRD patterns, meaning that the adsorbents were amorphous and/or highly dispersed [45,46]. In the fresh Na, K, and NRNa samples, some residual nitrates peaks were observed, which eventually disappeared upon reduction, as seen in FIG. 4. Furthermore, in the fresh DFMs patterns, the peaks located at 2θ°=37.2°, 43.3°, 62.9°, and 75.4° (JCPDS 00-047-1049) were ascribed to NiO. The existence of nickel aluminate (NiAl₂O₄) spinels was discarded due to the low calcination temperature used [47]. Small peaks of RuO₂ were also observed in the fresh samples (JCPDS 01-070-2662). No evidence of a Ni—Ru alloy was detected in the fresh DFMs.

Upon reduction at 800° C., as observed in FIG. 4, the CeO₂ peaks either disappeared or were significantly reduced. As a result, it was concluded that surface CeO₂ species were mainly present in the samples, which was in agreement with the H₂-TPR results [48]. No ceria aluminate (CeAlO₃) species were seen due to the reduction temperature being 800° C. [43]. Metallic Ni peaks were also detected in the reduced DFMs XRD patterns (JCPDS 01-070-1849). In contrast, metallic Ru peaks were not observed, which led us to believe that Ru was well dispersed and/or a Ni—Ru alloy was formed; however, due to the low Ru weight content (1 wt. %) compared to Ni (15 wt. %), no shift of Ni peaks took place in the XRD patterns [49].

### Example 4—Morphological Structure

The morphological structure of the fresh supported adsorbents and the DFMs was observed in SEM. This showed that after calcination, the materials were porous, which was in accordance with the BET results. The EDX mappings indicated that the materials were successfully impregnated as dispersed and homogenous materials were created. The high dispersion, porosity, and homogeneity of the DFMs were significant parameters that contributed to their impressive performance, as described later, due to the need to have a plethora of adsorption and catalytic sites in close proximity in order for them to work efficiently [24,50].

### Example 5—Reducibility profiles H₂-TPR

The reduction properties and interaction between the species of the supported adsorbents and the DFMs were observed in the H₂-TPR studies. FIG. 5 shows the H₂ signals of the DFMs and FIG. 6 the CH₄, H₂O, CO and CO₂ signals (m/z=15, 18, 28, 44) recorded during the H₂-TPR experiments. In FIG. 6, NR was used as a reference material [42].

As seen in FIG. 5, which presented similar peaks to the H₂O signal, the NRNa and NRK samples had a similar pattern since three distinct peaks were observed. The NRCa sample showed a broader reduction pattern up to 350° C., but two peaks were seen over that temperature. By observing the H₂O signals of the three DFMs in FIG. 6, it can be seen that the first peak at 200° C. corresponded to the reduction of Ru⁴⁺ to metallic Ru. These were located at a higher temperature compared to the reference material, indicating a better interaction of the Ru species with the other species [51]. Moreover, a broad peak located at 440° C. for the NRNa, another at 455° C. for the NRK, and another one at 520° C. for the NRCa were detected. In all those peaks, a shoulder at the lower temperature range was displayed, attributed to the medium-sized NiOx species interacting with the CeO₂ particles [52,53]. The slow decrease of the signal over 800° C. corresponded to the reduction of a small amount of bulk CeO₂ species, confirming the XRD results in FIG. 4, which showed that surface CeO₂ species were the main CeO₂ species in the samples.

A striking finding was the detection of CH₄ in the DFMs compared to NR, as seen in FIG. 6. By looking at the CO₂, H₂O, and H₂ signals, besides the CH₄, it can be seen that after their synthesis, these DFMs were able to adsorb CO₂ directly from the atmospheric air and convert it into CH₄ via the CO₂ methanation reaction. Therefore, Ni and Ru, which were already reduced by these temperatures (i.e. 440° C. for NRNa, 455° C. for NRK, and 520° C. for NRCa) were used to produce CH₄ and H₂O from the atmospheric CO₂ and the available H₂ from the H₂-TPR study. Desorption of CO₂ and generation of other products of CO₂ reduction were also detected mainly at low temperatures, in agreement with the CO₂-TPD results and the different types of basic sites, as presented later. Consequently, the main peak of the DFMs in the H₂O signal profiles corresponded to the H₂O from the methanation reaction, holding promise for using DFMs for flexible chemicals synthesis from CO₂ in the air.

### Example 6—Basicity Profiles CO₂-TPD

The CO₂-TPD was used so as to assess the basicity of the supported adsorbents and DFMs. In a CO₂-TPD profile, different types of basic sites may be present, which can be categorised into weak, medium, and strong ones. Weak basic sites are present up to 250° C., as they are unstable, while medium basic sites are decomposed from 250° C. to 700° C. Finally, the strong basic sites, which are the most stable ones, do not desorb the CO₂ until the temperature has reached 700° C. [54,55].

FIG. 7 demonstrates the CO₂-TPD results of DFMs and supported adsorbents, while FIG. 8 the results of the reference material. All the materials evidently displayed mainly weak and medium basic sites. By comparing the DFMs with the NR sample, it was noted that the addition of the adsorbents contributed to the formation of medium basic sites. Therefore, it was proved that the adsorbents were in a dispersed form rather than bulk species, meaning that the DFMs would behave like mid-temperature adsorbents and reversibly adsorb CO₂ at intermediate temperatures in agreement with literature [56]. Although the CO₂-TPD profiles of the DFMs had been mainly influenced by the supported adsorbents, it was observed that the addition of Ni and Ru to the supported adsorbents suppressed the medium basic sites. This meant that, to some extent, Ni and Ru covered a small amount of adsorption sites during impregnation [55]. Although based on the intensity of the signals, NRK displayed increased medium basic sites (300-700° C.), in agreement with its better activity at intermediate temperatures as described later on, NRNa and NRK samples had similar profiles to each other. As regards the NRCa sample, a different profile was observed with a smaller amount of weak basic sites and more medium-strong ones, in agreement with its CO₂ signal profile in the H₂-TPR study as well as the TGA study at high temperatures [55,57].

### Example 7—Adsorption and Desorption Studies in TGA

The main concern of selecting an adsorbent that carries out the CO₂ capture step at different temperatures is its feasibility of efficiently adsorbing the CO₂ in the 350-650° C. temperature range. CO₂ adsorption is an exothermic process and it is favoured at lower temperatures, which is why CO₂ methanation is the most studied reaction [25]. The use of mid-temperature adsorbents in the flexible DFMs scenario was therefore considered necessary. Five (5) cycles of CO₂ adsorption and desorption were performed at different temperatures and the results of the DFMs are presented in FIGS. 9 and 10, those of the supported adsorbents in FIGS. 11 and 12, and the ones of the reference material in FIG. 13. Table 2 (below) summarises the adsorption capacities of the DFMs at different temperatures based on their first cycle.

FIG. 9 shows that the designed materials were able to reversibly adsorb CO₂ in the temperature range of 350-650° C., making them the ideal choice for the flexible DFMs scenario. They all displayed relatively high adsorption capacities without the requirement of high regeneration temperatures as the desorption was carried out by an inert gas purge. Concerning the NRNa sample, a drop in the adsorption capacity was demonstrated in respect of temperature, although that was not the case at 650° C. A similar trend was also observed in the NRCa sample. A pattern was not easily detected in the NRK sample, but it was noted that it had the best performance at intermediate temperatures, in accordance with the CO₂-TPD results. As a general outcome of this study, NRNa seemed to perform best at low temperatures, NRK at intermediate temperatures, and NRCa at high temperatures, in agreement with other findings [55,57]. However, the adsorption capacities of all DFMs at 350° C. and 650° C., which were the temperatures used for their feasibility study, were not massively different, explaining the success of all the materials as flexible DFMs. Indeed, the fact that the adsorption capacity at 650° C. was even better at 2nd-5th cycles than that at 350° C. was astonishing.

As can be seen in FIG. 9, a substantial amount of CO₂ was adsorbed during the first cycle, which was not the case in the supported adsorbents or the switchable catalyst. Especially at 350° C., the adsorption capacity of the DFMs was higher than the supported adsorbents and the catalyst together, proving that a synergy between those materials took place, which enhanced the CO₂ adsorption capacity. In other words, CO₂ was not adsorbed only onto the adsorbents and the catalysts, a fact that has already been established in literature [58,59]. Consequently, these results demonstrate that when adsorbents and catalysts are in close proximity, they benefit from each other. The findings showcase the importance of having one material performing both the CO₂ adsorption and reduction, rather than two different materials being mixed together. Additionally, as the role of CeO₂ in CO₂ adsorption is not yet understood, further mechanistic studies need to be done in the future. Nevertheless, it was firmly concluded that a small quantity of CO₂ was used to oxidise the reduced Ce species and form CO, probably explaining the small amounts of CO being formed during the CO₂ capture step in their feasibility testing. After all, Ce is well known for its excellent redox properties [35,60].

FIGS. 10, 12 and 13 show the amount of CO₂ adsorbed per mg of sample during those five cycles at different temperatures. All the materials demonstrated that the adsorption was performed in two steps. An initial fast adsorption of CO₂ took place, accounting for the substantial weight gain at the beginning of each capture cycle. The second step was a slower one, evident from the change in slope. In addition, it was observed that the supported adsorbents were able to reach equilibrium and reversibly adsorb CO₂ onto the adsorbents' sites, meaning that they had managed to adsorb and desorb the same CO₂ amounts, which was not the case with the DFMs. An interesting observation was that there were different slopes in the CO₂ adsorption steps at 650° C. compared to those at 350° C., proving that a different adsorption mechanism had taken place. However, it was found that the change in slope was not attributed to the adsorbents, but to the Ni—Ru species, as can be seen by comparing FIGS. 10, 12 and 13. This result also showcased that the catalytic component of the DFMs contributed to the CO₂ adsorption too and highlighted the importance of a synergy between these two components. Overall, it was concluded that the materials displayed an increased complexity, and thus further mechanistic studies could be carried out in the future so as to fully understand the adsorption as well as the reaction mechanisms.

TABLE 2
Summary of adsorption capacities of DFMs at
different temperatures

| Temperature | Adsorption Capacity mol/kg | | |
|---|---|---|---|
| (° C.) | NRNa | NRK | NRCa |
| 350 | 0.49 | 0.29 | 0.39 |
| 450 | 0.32 | 0.17 | 0.17 |
| 550 | 0.14 | 0.15 | 0.20 |
| 650 | 0.16 | 0.18 | 0.26 |

### Example 8—Feasibility Study of Flexible DFMs

After proving that the materials had been able to adsorb CO₂ at different temperatures, the feasibility study of their flexibility scenario was carried out. In line to previous findings [42,61], it was decided to perform the CO₂ methanation reaction first, followed by the RWGS and DRM reactions, by carrying out a CO₂-capture step before each reaction. An N₂ purge step was used in between the capture and the reaction steps until the CO₂ reading in the gas analyser reached zero so as to prove that the produced gases were formed from the captured CO₂. An N₂ purge step was also used following the CO₂ methanation reaction and prior to the heating-up step for 10 mins, as well as after the RWGS reaction and before the CO₂ capture step in order to obtain zero readings in the gas analyser. The percentage of CO₂ in the mixture was 10% so as to simulate the CO₂ content in effluent streams [25]. FIG. 14 shows the amount of CO₂ desorbed and products formation (CH₄, CO or H₂) in each reaction for the three DFMs and FIGS. 15, 16 and 17 show the volumetric flow rates of all gases vs time plots of NRNa, NRK, and NRCa, respectively.

FIG. 14 reveals the success of flexible DFMs. In all cases, the CO₂ was captured at the desired temperature and was subsequently converted into CH₄, CO, and syngas, depending on the temperature and the co-reactant used. It was demonstrated that all DFMs had 100% selectivity in all three reactions and the formation of by-products was prevented. A small amount of CO₂ was desorbed during all reactions. However, it seemed that this was not temperature dependent, because it was found out that approximately 10% of CO₂ was desorbed in both CO₂ methanation and DRM. This meant that this 10% of the adsorption sites was not in proximity with the catalytic sites, so the adsorbed CO₂ ended up being desorbed. No CO₂ was detected during the heating up step, proving that the entire amount of CO₂ adsorbed had either been converted into CH₄ or was desorbed.

Furthermore, it was observed that all DFMs were very active in DRM. NRK was the best DFM in CO₂ methanation followed by NRCa and NRNa. In both RWGS and DRM, NRCa was the best DFM and after that, it was NRK and NRNa. It was concluded that the DFMs performance strongly depended on the materials basicity and their CO₂ desorption ability at the selected temperature, as observed in the CO₂-TPD profiles. Additionally, it was proved that the proximity of the adsorption and catalytic sites was a more vital parameter compared to the adsorption capacity. This was because even if NRNa had the highest CO₂ capacity at 350° C., it had the worst performance in CO₂ methanation reaction indicating an unsatisfactory interaction between the Na and Ni—Ru species. Overall, their performance could be generalised as NRCa>NRK>NRNa.

In terms of product signals as observed in FIGS. 15-17, an initial spike in the products was detected, demonstrating a fast reaction between the captured CO₂ and the in-excess co-reactant, followed by a slower decrease in their signals. Additionally, it was observed that even though the reaction step lasted for 20 mins, the reaction steps had taken place in the first 10 min, allowing space for further optimisation of the process by minimising the reaction step time. It is worth noting, however, that in order for two reactors to run in parallel in the DFM technology, the CO₂ capture and reduction steps need to have the same duration and if this is not the case, alternative configurations are required.

An interesting finding was that of the CO formation during the capture steps, probably showing that CO₂ reacted with Ce³⁺, or even with Ni and Ru species and formed CO. These species were re-reduced when H₂ was either flowing or being generated in the subsequent steps [35,41]. This would in turn mean that the materials were able to sequentially be oxidised and reduced throughout the duration of the experiment without any structure alteration, as proved by the post-characterisation of the spent materials. Another explanation of the CO formation during the capture step would have been the occurrence of the reverse Boudouard reaction between the CO₂ and some carbon species, resulting in CO production. In addition to the CO formation, there was also an amount of H₂ produced during the capture step. This could have happened because of H₂ being chemisorbed in a previous step and hence being displaced by CO₂ and/or the oxidation of Ce³⁺ species with H₂O to produce Ce⁴⁺ and H₂. To sum up, it was demonstrated that several reactions may have taken place during the CO₂ capture.

By observing the CO and H₂ profiles of the NRNa during RWGS, an odd result emerged. It appeared that even when no CO and H₂ were produced during that reaction, the H₂ signal decreased and no CH₄ was formed. However, during the subsequent CO₂ capture step, these gases were indeed detected. Therefore, it was assumed that there had not been enough heat produced during that step so as to release the products of the endothermic RWGS. Once a small amount of heat was produced during the next exothermic adsorption step, these gases were able to be released, and thus detected by the gas analyser. As a result, the DFM's sites were once again free to adsorb CO₂ during the capture step.

During DRM, the DFMs were able to initially convert the captured CO₂ into syngas, as illustrated by the CO and H₂ signals. However, methane cracking was observed for all DFMs as a significant amount of CH₄ was used and H₂ was produced. This was anticipated because the captured CO₂ had initially been converted into syngas and, as time went by and its availability was limiting, the CH₄ was decomposed into carbon and H₂. This phenomenon had already been reported during the post-breakthrough stage [39]. As a result, the H₂/CO ratio was higher than its stoichiometric value of 1 and a H₂ rich syngas was produced which could be useful in CO₂ applications that require a higher H₂/CO ratio. A good strategy for limiting the carbon formation would have been to decrease the DRM duration, as it had taken place predominantly in the first 5 to 10 mins, because allowing this step to occur for longer would serve no purpose. Nevertheless, previous reports [38-40] demonstrated that the carbon formed during the DRM reaction was able to be regenerated during the subsequent CO₂ capture step via the endothermic reverse Boudouard reaction. This phenomenon was not observed here as the DRM reaction was the last step in this set of experiments. However, the occurrence of the reverse Boudouard reaction would have been expected for these materials too if a CO₂ capture step had occurred after the DRM step.

### Example 9—Post Reaction Characterisation

As shown in FIGS. 15-17, CH₄ cracking took place in the feasibility study of the flexible DFMs that resulted in the formation of coke, and hence XRD and TGA were carried out on the spent catalysts to qualify and quantify the carbon species.

FIG. 18 shows the XRD patterns of the post-reaction NRNa, NRK, and NRCa samples. The patterns were similar to their reduced ones presented in FIG. 4, displaying the characteristic peaks of Ni, CeO₂, and Al₂O₃. No carbon peak was observed in the spent NRNa and NRK XRD profiles, meaning that the type of carbon formed was soft with a poorer degree of crystallinity. However, this was not the case with the NRCa, as a carbon peak at 2θ°=26 was observed, signifying the formation of a harder carbon requiring higher regeneration temperatures. A slight shift of the Ni peak to the left appeared on the NRNa and NRCa samples indicating the formation of a NiRu alloy, which was not the case for their reduced materials. [22]. Hence, it was concluded that a NiRu alloy was formed during reduction even if it was not evident from their XRD patterns.

Thermogravimetric analysis was also used to quantify the carbon formed during the DRM reaction. In order to accurately measure the amount of carbon, ex situ reduced DFMs were also tested in the same conditions. Therefore, the weight loss due to the atmospheric CO₂ and moisture adsorption and material degradation, as well as the weight gain due to the oxidation of the materials, were also taken into account. The results are presented in FIG. 19. In all the samples, an initial weight loss took place up to 200° C. because of the weakly adsorbed atmospheric CO₂ and moisture. A higher weight loss was seen in the reduced materials because they were able to absorb a higher amount of atmospheric impurities. Moreover, a weight gain was observed at the 300-400° C. temperature range, which was associated with the oxidation of the remaining Ni and Ru particles. At temperatures higher than 400° C., a significant weight loss was detected, showing the carbon oxidation. In agreement with the XRD results, the coke oxidation was completed by 500° C. for the NRNa and NRK samples, while a higher temperature was needed for the NRCa sample, confirming the existence of softer carbon species in the former samples and of harder ones in the latter. The formation of soft carbon was an intriguing result as it will likely allow an easier regeneration process in the future. Overall, the amount of carbon formed during the DRM reaction was 0.083 gC/gSample for NRNa, 0.072 gC/gSample for NRK and 0.075 gC/gSample for NRCa, showing a similar extent of CH₄ cracking for the three DFMs.

### Example 10—CH₄-Temperature Programmed Surface Reaction (CH₄-TSPR)

A CH₄-TSPR was conducted on the fresh NiRuCa sample in a fixed bed quartz reactor. The data were logged by using the Quadera software package and the signals of gases were observed in an online mass spectrometer (Omni-Star GSD 320). In this experiment, 30 mg of fresh (after calcination) NiRuCa was used and the temperature was increased from room temperature to 950° C. (10° C./min) with a pure CH₄ feed of 30 ml/min. No N₂ was used in the feed in order that mass to charge ratio (m/z) of 28 to be solely attributed to CO.

The aim was to observe the fresh DFM performance in a CH₄ environment, similar to the H₂-TPR experiment, and identify opportunities for a potential temperature reduction of the high-temperature reactions. This would ultimately be translated into energy requirements reduction, minimising the operating costs in a potential scale-up of the switchable DFMs.

FIG. 20 shows the CH₄-TPSR results. Overall, it was demonstrated that the DFM's oxidation (since the sample used was after calcination) did not shut down its activity for DRM which was remarkable. In agreement with the H₂-TPR results, atmospheric CO₂ was adsorbed onto the DFM and was gradually released until a peak at 400° C. was observed. A peak at higher temperature analogous to the H₂-TPR and CO₂-TPD results was not identified, meaning that CO₂ was fully consumed during low temperature reaction. A CO peak was also seen at this temperature (400° C.). Additionally, CH₄ cracking took place starting at 350-400° C. and peaked at 635° C., as observed from the H₂ and CH₄ signals. Therefore, it was shown that in a CH₄-rich environment and in the temperature range of 400-600° C., CH₄ cracking supplied the H₂ needed for the reduction of Ni and Ru which was feasible at these temperatures. Thus, DRM was able to take place because of the available CH₄ and atmospheric CO₂. However, after the consumption of CO₂, excessive CH₄ cracking took place, which was expected based on our isothermal experiment results.

Based on these results, it was confirmed that a DRM temperature reduction to 400° C. could be feasible. It should be kept in mind that the DFMs operate under dynamic conditions, meaning that a deviation from the well-known steady state conditions is possible. The initial temperature of RWGS and DRM, i.e. 650° C. was considered useful for the validation study since higher temperatures are more representative of the current industrial reformers that operate close to equilibrium conversions at 900-1000° C. Investigating the DFMs behaviour at a temperature higher than 650° C. was considered to be unnecessary, because the DFMs had a 100% selectivity in these reactions. In fact, it would have had a negative effect due to reduced CO₂ adsorption and sintering, let alone the increase of operating costs in a potential scale up.

### CONCLUSIONS

The inventors have designed a series of advanced universal materials for the integrated CO₂ capture and conversion. The combination of an adsorbent and a switchable catalyst gave birth to the flexible DFMs that were able to capture CO₂ at various temperatures and, depending on the reaction conditions, to convert it into different added-value chemicals through the CO₂ methanation, RWGS, and DRM catalytic upgrading routes. Their proof of existence is a milestone in the development of carbon negative technologies to combat the increased CO₂ emissions and the subsequent global warming. Flexible DFMs can be adapted to the current infrastructure as a CCU unit, while offering a solution to the fragile energy sector.

The designed DFMs of this project were proved to be highly dispersed porous materials and their species, located in close proximity, showed a high degree of interaction with each other. The H₂-TPR experiments demonstrated that these materials were able to adsorb the atmospheric CO₂ and, upon Ni and Ru reduction, to convert it into synthetic natural gas. The CO₂ adsorption-desorption studies of the selected DFMs surprisingly proved that they were able to reversibly adsorb CO₂ at different temperatures, making them ideal for a flexible DFM scenario. A synergy between the species also took place, contributing to their increased adsorption capacities. Overall, the CO₂ adsorption capacities of the DFMs were in accordance with their basicity profiles, as revealed by the CO₂-TPD results.

The feasibility study of performing CO₂ capture and then either CO₂ methanation, RWGS or DRM reactions was successful. The DFMs were capable of adsorbing CO₂ from a CO₂-containing stream and then, depending on the reaction temperature and the co-reactant used, CO₂ was converted into CH₄, CO, and syngas, respectively. The results showed that flexible DFMs exist and can be used as a tool in green technologies in the future, even if the optimisation of materials and conditions is necessary to combat issues, like the formation of coke during DRM. This work is undoubtedly the beginning of the development of advanced universal materials, and its findings are unambiguously a step forward in the fight against CO₂ emissions globally.

### Methods

### Characterisation

The Brunauer-Emmett-Teller (BET) equation and the Barett-Joyner-Halenda (BJH) methods were used to obtain the specific surface area and pore volume of the materials. Initially, degassing at 250° C. in vacuum for 4 hours took place and then, the textural properties of the materials were determined by nitrogen adsorption-desorption measurements at −195° C. in a Micrometrics 3Flex apparatus.

X-ray diffraction was performed on fresh supported adsorbents and on fresh, reduced, and spent DFMs in a X′Pert Powder from PANalytical apparatus. The diffraction patterns were obtained at 30 mA and 40 kV by using Cu Kα radiation (λ=0.154 nm). The 2θ° angle was increased every 450s by 0.05° in the range of 10-90°.

Scanning electron microscopy was carried out on the fresh supported adsorbents and DFMs by using a JEOL JSM-7100F instrument, which also had an Energy Dispersive X-ray Spectroscope (EDS) analyser. Carbon paint was used to fix the samples to the holder and coating was conducted to eliminate the charging effects.

H₂-Temperature Programmed Reduction (TPR) was performed on the fresh supported adsorbents and DFMs in fixed bed quartz reactor. The data were logged by using the Quadera software package and the H₂ consumption was observed in an online mass spectrometer (Omni-Star GSD 320). A 10% H₂/Ar mixture with a total flow of 50 ml/min was passed through the reactor with 50 mg of sample while the temperature was raised from room temperature to 950° C. with 10° C./min rate.

CO₂-Temperature Programmed Reduction (CO₂-TPD) was carried out on the same equipment as H₂-TPR. In the CO₂-TPD experiments, 50 mg of sample were initially reduced in situ at 800° C. with a 10% H₂/N₂ mixture and a total flow of 50 ml/min (10° C./min). After they had cooled down to 40° C. with a N₂ purge, a flow 50 ml/min of a 10% CO₂/N₂ mixture was used for 45 mins to saturate the samples. Subsequently, 50 ml/min of N₂ were flowed through the reactor to remove the weakly adsorbed CO₂ for 30 mins and, finally, the temperature was raised to 800° C. at a 10° C./min rate. The mass to charge ratio (m/z) of 44 corresponding to CO₂ was recorded during the temperature increase.

Thermogravimetric analysis (TGA) was performed on the reduced and spent DFMs after the reactor performance tests in a SDT650 apparatus from TA Instruments in order to measure the amount of carbon depositions. A flow of 100 mL/min of air was employed while the temperature was raised from room temperature to 950° C. at a 10° C./min rate.

### Performance Testing

### Adsorption and Desorption Study at Different Temperatures

A SDT650 apparatus from TA Instruments was used so as to understand the adsorption and desorption behaviour of the materials at different temperatures. 10-15 mg of a reduced sample was used in each run. Firstly, the temperature was increased from room temperature to 150° C. with 100 mL/min of Ar at a 10° C./min rate and was held at that temperature for 30 mins in order for all the weakly adsorbed gases to be desorbed. Next, it was raised to 350° C., 450° C., 550° C., or 650° C. with the same Ar flow and temperature rate as before. Subsequently, the temperature was stabilised at the desired level for 200 mins, allowing 5 cycles to be conducted, each of which consisted of 20 mins of CO₂ adsorption and 20 mins of CO₂ desorption. In each adsorption step, 20 ml/min of CO₂ and 100 ml//min of Ar were used and in each desorption step, 100 ml/min of Ar. The test was carried out on the reduced supported adsorbents and DFMs. The reduction was performed ex situ at 800° C. for 1 hr at a 50 ml/min total flow rate of a 10% H₂/N₂ mixture.

### Feasibility Study of Flexible DFMs

The three DFMs were tested in a tubular fixed bed quartz reactor (0.5 in OD) at atmospheric pressure and were supported on a quartz wool bed. The volumetric percentages of CO₂, CH₄, CO, and H₂ in the outlet stream were monitored, using an ABB AO2020 online gas analyser, which was placed after the water was condensed and separated in a chiller. A bubble meter, which was a vertical tube of a known volume, was used to allow the accurate measurement of the total volumetric flow rate by simply measuring the time spent for a bubble to reach that known volume. Two different modes of experimental set up were used in those feasibility experiments: the ‘reactor’ mode and the ‘bypass’ mode. In the former, the desired gases were passing through the reactor, whereas in the latter, they were bypassing it, going to the condenser and then to the ABB gas analyser or the bubble meter. A simplified process diagram is shown in FIG. 1.

Initially, 0.25 g of sample were reduced in situ at 800° C. for 1 hr at a 50 ml/min total flow rate of a 10% H₂/N₂ mixture (10° C./min). Then, the temperature was decreased to 350° C. with a N₂ purge stream in order to perform a cycle of CO₂ capture-N₂ purge-CO₂ methanation. The CO₂ capture step was performed in the ‘reactor’ mode with a 10% CO₂/N₂ mixture of 50 ml/min total flow rate for 20 mins. After the 20 mins had elapsed, a 10 mins N₂ purge step was carried out, resulting in the removal of the weakly adsorbed CO₂ and obtaining a zero CO₂ reading in the gas analyser. Subsequently, in the ‘bypass’ mode, a mixture containing 10% of H₂ in N₂ was flowing through the system until its reading in the analyser was stabilised and then in the ‘reactor’ mode, the reaction step was performed for 20 mins. Once the CO₂ capture-N₂ purge-CO₂ methanation cycle was carried out, the temperature was increased to 650° C. at a 10° C./min rate. A cycle of CO₂ capture-N₂ purge-RWGS and a cycle of CO₂ capture-N₂ purge-DRM followed as described above, but in the case of DRM, CH₄ instead of H₂ was used in the reaction step.

It is worth noting that the N₂ flow remained the same throughout the experiment as in all the CO₂ capture and reaction steps, it represented 90% of the mixture, i.e. 45 mL/min and its exact flow rate was measured at the beginning of the experiment. This meant that when the total flow rates in CO₂ capture and reaction steps were measured with the bubble meter, the exact flow rates of CO₂ and co-reactant, either H₂ or CH₄, were calculated by subtracting the known N₂ flow rate from the corresponding total flow rate. Moreover, all the total volumetric flow rate measurements and the stabilisation of the gases percentage were performed in the ‘bypass’ mode to make sure that the DFMs were exposed to the desired gases only during the CO₂ capture, N₂ purge, and reaction steps.

In addition, the percentages of CO₂, CH₄, CO, and H₂ in the outlet stream shown in the gas analyser were recorded every 5 sec throughout the experiment and it was assumed that the remaining volume percentage in the mixture was N₂. Hence, the volumetric flow rates of all gases were calculated according to the following formula, where the brackets represent the volumetric percentage of each gas, i.e. CO₂, CH₄, CO, and H₂, the F the flow rate (mL/min), and the subscript ‘i’ the respective gas. The amount of products (in mL) was calculated based on the area under the curve at a flow rate (mL/min) vs time (min) graph.

$${F}_{i} = \frac{[ i ]}{[ {N}_{2} ]} \times {F}_{{N}_{2}}$$

### REFERENCES

[1] IEA. Global energy review-CO₂ emissions 2021. https://www.iea.org/reports/global-energy-review-2021/co2-emissions (accessed April 4,2022).
[2] Zhang Q, Pastor-Pérez L, Gu S, Reina T R. Transition metal carbides (TMCS) catalysts for gas phase CO₂ upgrading reactions: A comprehensive overview. Catalysts 2020; 10:1-23. https://doi.org/10.3390/catal10090955.
[3] Trends in Atmospheric Carbon Dioxide. Noaa/Esrl 2016: http://www.esrl.noaa.gov/gmd/ccgg/trends/global.ht. ftp://aftp.cmdl.noaa.gov/products/trends/co2/co2_mm_gl.txt (accessed Jul. 6, 2021).
[4] United Nations Framework Convention on Climate Change. Adoption of the Paris Agreement. Paris: 2015.
[5] NASA Global Climate Change Vital Signs of the Planet 2021. https://climate.nasa.gov/vital-signs/carbon-dioxide/(accessed Jul. 6, 2021).
[6] Rogelj J, Den Elzen M, Höhne N, Fransen T, Fekete H, Winkler H, et al. Paris Agreement climate proposals need a boost to keep warming well below 2° C. Nature 2016; 534:631-9. https://doi.org/10.1038/nature18307.
[7] Pires J C M, Martins F G, Alvim-Ferraz M C M, Simões M. Recent developments on carbon capture and storage: An overview. Chem Eng Res Des 2011; 89:1446-60. https://doi.org/10.1016/j.cherd.2011.01.028.
[8] Samanta A, Zhao A, Shimizu G K H, Sarkar P, Gupta R. Post-combustion CO 2 capture using solid sorbents: A review. Ind Eng Chem Res 2012; 51:1438-63. https://doi.org/10.1021/ie200686q.
[9] Damen K, Troost M Van, Faaij A, Turkenburg W. A comparison of electricity and hydrogen production systems with CO 2 capture and storage. Part A: Review and selection of promising conversion and capture technologies. Prog Energy Combust Sci 2006; 32:215-46. https://doi.org/10.1016/j.pecs.2005.11.005.
[10] Rao A B, Rubin E S. A technical, economic, and environmental assessment of amine-based CO₂ capture technology for power plant greenhouse gas control. Environ Sci Technol 2002; 36:4467-75. https://doi.org/10.1021/es0158861.
[11] Al-Mamoori A, Krishnamurthy A, Rownaghi A A, Rezaei F. Carbon Capture and Utilization Update. Energy Technol 2017; 5:834-49. https://doi.org/10.1002/ente.201600747.
[12] Mondal M K, Balsora H K, Varshney P. Progress and trends in CO₂ capture/separation technologies: A review. Energy 2012; 46:431-41. https://doi.org/10.1016/j.energy.2012.08.006.
[13] Wang W, Wang S, Ma X, Gong J. Recent advances in catalytic hydrogenation of carbon dioxide. Chem Soc Rev 2011; 40:3703-27. https://doi.org/10.1039/c1cs15008a.
[14] Najafabadi A T. CO 2 chemical conversion to useful products: An engineering insight to the latest advances toward sustainability 2013:485-99. https://doi.org/10.1002/er.
[15] Pakhare D, Spivey J. A review of dry (CO₂) reforming of methane over noble metal catalysts. Chem Soc Rev 2014. https://doi.org/10.1039/c3cs60395d.
[16] Bradford M C J, Vannice M A. CO₂ reforming of CH₄. Catal Rev-Sci Eng 1999; 41:1-42. https://doi.org/10.1081/CR-100101948.
[17] Usman M, Wan Daud W M A, Abbas H F. Dry reforming of methane: Influence of process parameters-A review. Renew Sustain Energy Rev 2015; 45:710-44. https://doi.org/10.1016/j.rser.2015.02.026.
[18] Liu M, Yi Y, Wang L, Guo H, Bogaerts A. Hydrogenation of carbon dioxide to value-added chemicals by heterogeneous catalysis and plasma catalysis. Catalysts 2019; 9. https://doi.org/10.3390/catal9030275.
[19] Yang H, Xu Z, Fan M, Gupta R, Slimane R B, Bland A E, et al. Progress in carbon dioxide separation and capture: A review. J Environ Sci 2008; 20:14-27. https://doi.org/10.1016/S1001-0742 (08) 60002-9.
[20] Daza Y A, Kuhn J N. CO₂ conversion by reverse water gas shift catalysis: Comparison of catalysts, mechanisms and their consequences for CO₂ conversion to liquid fuels. RSC Adv 2016; 6:49675-91. https://doi.org/10.1039/c6ra05414e.
[21] Frontera P, Macario A, Ferraro M, Antonucci P L. Supported catalysts for CO₂ methanation: A review. Catalysts 2017; 7:1-28. https://doi.org/10.3390/catal7020059.
[22] Lange F, Armbruster U, Martin A. Heterogeneously-Catalyzed Hydrogenation of Carbon Dioxide to Methane using RuNi Bimetallic Catalysts. Energy Technol 2015; 3:55-62. https://doi.org/10.1002/ente.201402113.
[23] Gao J, Liu Q, Gu F, Liu B, Zhong Z, Su F. Recent advances in methanation catalysts for the production of synthetic natural gas. RSC Adv 2015; 5:22759-76. https://doi.org/10.1039/c4ra16114a.
[24] Duyar M S, Treviño M A A, Farrauto R J. Dual function materials for CO₂ capture and conversion using renewable H₂. Appl Catal B Environ 2015; 168-169:370-6. https://doi.org/10.1016/j.apcatb.2014.12.025.
[25] Merkouri L P, Reina T R, Duyar M S. Closing the Carbon Cycle with Dual Function Materials. Energy and Fuels 2021; 35:19859-80. https://doi.org/10.1021/acs.energyfuels.1c02729.
[26] Omodolor I S, Otor H O, Andonegui J A, Allen B J, Alba-Rubio A C. Dual-Function Materials for CO 2 Capture and Conversion: A Review. Ind Eng Chem Res 2020; 59:17612-31. https://doi.org/10.1021/acs.iecr.0c02218.
[27] Sun S, Sun H, Williams P T, Wu C. Recent advances in integrated CO 2 capture and utilization: a review. Sustain Energy Fuels 2021; 5:4546-59. https://doi.org/10.1039/dise00797a.
[28] Melo Bravo P, Debecker D P. Combining CO₂ capture and catalytic conversion to methane. Waste Dispos Sustain Energy 2019; 1:53-65. https://doi.org/10.1007/s42768-019-00004-0.
[29] Arellano-Treviño M A, Kanani N, Jeong-Potter C W, Farrauto R J. Bimetallic catalysts for CO₂ capture and hydrogenation at simulated flue gas conditions. Chem Eng J 2019; 375:121953. https://doi.org/10.1016/j.cej.2019.121953.
[30] Wang S, Schrunk E T, Mahajan H, Farrauto R J. The role of ruthenium in CO₂ capture and catalytic conversion to fuel by dual function materials (DFM). Catalysts 2017; 7:1-13. https://doi.org/10.3390/catal7030088.
[31] Duyar M S, Wang S, Arellano-Treviño M A, Farrauto R J. CO₂ utilization with a novel dual function material (DFM) for capture and catalytic conversion to synthetic natural gas: An update. J CO₂ Util 2016; 15:65-71. https://doi.org/10.1016/j.jcou.2016.05.003.
[32] Jeong-Potter C, Farrauto R. Feasibility Study of Combining Direct Air Capture of CO₂ and Methanation at Isothermal Conditions with Dual Function Materials. Appl Catal B Environ 2020:119416. https://doi.org/10.1016/j.apcatb.2020.119416.
[33] Hu L, Urakawa A. Continuous CO₂ capture and reduction in one process: CO₂ methanation over unpromoted and promoted Ni/ZrO₂. J CO₂ Util 2018; 25:323-9. https://doi.org/10.1016/j.jcou.2018.03.013.
[34] Sun S, Lv Z, Qiao Y, Qin C, Xu S, Wu C. Integrated CO₂ capture and utilization with CaO-alone for high purity syngas production. Carbon Capture Sci Technol 2021; 1:100001. https://doi.org/10.1016/j.ccst.2021.100001.
[35] Sun H, Wang J, Zhao J, Shen B, Shi J, Huang J, et al. Dual functional catalytic materials of Ni over Ce-modified CaO sorbents for integrated CO₂ capture and conversion. Appl Catal B Environ 2019; 244:63-75. https://doi.org/10.1016/j.apcatb.2018.11.040.
[36] Bobadilla L F, Riesco-García J M, Penelas-Perez G, Urakawa A. Enabling continuous capture and catalytic conversion of flue gas CO₂ to syngas in one process. J CO₂ Util 2016; 14:106-11. https://doi.org/10.1016/j.jcou.2016.04.003.
[37] Shao B, Hu G, Alkebsi K A M, Ye G, Lin X, Du W, et al. Heterojunction-redox catalysts of Fe: XCoyMg10CaO for high-temperature CO₂capture and in situ conversion in the context of green manufacturing. Energy Environ Sci 2021; 14:2291-301. https://doi.org/10.1039/doee03320k.
[38] Jo S, Lee J H, Woo J H, Kim T-Y, Ryu H-J, Hwang B, et al. Coke-promoted Ni/CaO catal-sorbents in the production of cyclic CO and syngas. Sustain Energy Fuels 2022; 6:81-8. https://doi.org/10.1039/d1se01136g.
[39] Kim S M, Abdala P M, Broda M, Hosseini D, Coperet C, Müller C. Integrated CO₂ Capture and Conversion as an Efficient Process for Fuels from Greenhouse Gases. ACS Catal 2018; 8:2815-23. https://doi.org/10.1021/acscatal.7b03063.
[40] Tian S, Yan F, Zhang Z, Jiang J. Calcium-looping reforming of methane realizes in situ CO₂ utilization with improved energy efficiency. Sci Adv 2019; 5. https://doi.org/10.1126/sciadv.aav5077.
[41] Jo S Bin, Woo J H, Lee J H, Kim T Y, Kang H I, Lee S C, et al. CO₂green technologies in CO₂capture and direct utilization processes: methanation, reverse water-gas shift, and dry reforming of methane. Sustain Energy Fuels 2020; 4:5543-9. https://doi.org/10.1039/d0se00951b.
[42] Merkouri L-P, le Sache E, Pastor-Perez L, Duyar M S, Reina T R. Versatile Ni—Ru catalysts for gas phase CO 2 conversion: Bringing closer dry reforming, reverse water gas shift and methanation to enable end-products flexibility. Fuel 2022; 315. https://doi.org/10.1016/j.fuel.2021.123097.
[43] Le Sache E, Johnson S, Pastor-Pérez L, Horri B A, Reina T R. Biogas upgrading via dry reforming over a Ni—Sn/CeO₂—Al₂O₃ catalyst: Influence of the biogas source. Energies 2019; 12. https://doi.org/10.3390/en12061007.
[44] Tao K, Shi L, Ma Q, wang D, Zeng C, Kong C, et al. Methane reforming with carbon dioxide over mesoporous nickel-alumina composite catalyst. Chem Eng J 2013; 221:25-31. https://doi.org/10.1016/j.cej.2013.01.073.
[45] Wang S, Farrauto R J, Karp S, Jeon J H, Schrunk E T. Parametric, cyclic aging and characterization studies for CO₂ capture from flue gas and catalytic conversion to synthetic natural gas using a dual functional material (DFM). J CO₂ Util 2018; 27:390-7. https://doi.org/10.1016/j.jcou.2018.08.012.
[46] Bermejo-López A, Pereda-Ayo B, González-Marcos J A, González-Velasco J R. Mechanism of the CO₂ storage and in situ hydrogenation to CH₄. Temperature and adsorbent loading effects over Ru—CaO/Al₂O₃ and Ru—Na₂CO₃/Al₂O₃ catalysts. Appl Catal B Environ 2019; 256:117845. https://doi.org/10.1016/j.apcatb.2019.117845.
[47] Bermejo-López A, Pereda-Ayo B, González-Marcos J A, González-Velasco J R. Alternate cycles of CO₂storage andin situhydrogenation to CH₄on Ni—Na₂CO₃/Al₂O₃: influence of promoter addition and calcination temperature. Sustain Energy Fuels 2021; 5:1194-210. https://doi.org/10.1039/d0se01677b.
[48] Stroud T, Smith T J, Le Sache E, Santos J L, Centeno M A, Arellano-Garcia H, et al. Chemical CO₂ recycling via dry and bi reforming of methane using Ni—Sn/Al₂O₃ and Ni—Sn/CeO₂—Al₂O₃ catalysts. Appl Catal B Environ 2018; 224:125-35. https://doi.org/10.1016/j.apcatb.2017.10.047.
[49] Mierczynski P, Stępińska N, Mosinska M, Chalupka K, Albinska J, Maniukiewicz W, et al. Hydrogen production via the oxy-steam reforming of Ing or methane on ni catalysts. Catalysts 2020; 10. https://doi.org/10.3390/catal10030346.
[50] Zheng Q, Farrauto R, Chau Nguyen A. Adsorption and Methanation of Flue Gas CO₂ with Dual Functional Catalytic Materials: A Parametric Study. Ind Eng Chem Res 2016; 55:6768-76. https://doi.org/10.1021/acs.iecr.6b01275.
[51] Valdes-Martínez O U, Suarez-Toriello V A, Reyes J A d. los, Pawelec B, Fierro J L G. Support effect and metals interactions for NiRu/Al₂O₃, TiO₂ and ZrO₂ catalysts in the hydrodeoxygenation of phenol. Catal Today 2017; 296:219-27. https://doi.org/10.1016/j.cattod.2017.04.007.
[52] Pastor-Perez L, Buitrago-sierra R, Sepulveda-Escribano A. CeO₂-promoted Ni/activated carbon catalysts for the water-gas shift (WGS reaction. Int J Hydrogen Energy 2014; 9:17589-99. https://doi.org/10.1016/j.ijhydene.2014.08.089.
[53] Pastor-Pérez L, Sepúlveda-Escribano A. Multicomponent NiSnCeO2/C catalysts for the low-temperature glycerol steam reforming. Appl Catal A Gen 2017; 529:118-26. https://doi.org/10.1016/j.apcata.2016.10.022.
[54] Pan Q, Peng J, Sun T, Wang S, Wang S. Insight into the reaction route of CO₂ methanation: Promotion effect of medium basic sites. Catal Commun 2014; 45:74-8. https://doi.org/10.1016/j.catcom.2013.10.034.
[55] Bermejo-López A, Pereda-Ayo B, González-Marcos J A, González-Velasco J R. Ni loading effects on dual function materials for capture and in-situ conversion of CO₂ to CH₄ using CaO or Na₂CO₃. J CO₂ Util 2019; 34:576-87. https://doi.org/10.1016/j.jcou.2019.08.011.
[56] Gruene P, Belova A G, Yegulalp T M, Farrauto R J, Castaldi M J. Dispersed calcium oxide as a reversible and efficient CO₂sorbent at intermediate temperatures. Ind Eng Chem Res 2011; 50:4042-9. https://doi.org/10.1021/ie102475d.
[57] Cimino S, Boccia F, Lisi L. Effect of alkali promoters (Li, Na, K) on the performance of Ru/Al₂O₃ catalysts for CO₂ capture and hydrogenation to methane. J CO₂ Util 2020; 37:195-203. https://doi.org/10.1016/j.jcou.2019.12.010.
[58] Proaño L, Tello E, Arellano-Trevino M A, Wang S, Farrauto R J, Cobo M. In-situ DRIFTS study of two-step CO 2 capture and catalytic methanation over Ru, “Na 2 O”/Al 2 O 3 Dual Functional Material. Appl Surf Sci 2019; 479:25-30. https://doi.org/10.1016/j.apsusc.2019.01.281.
[59] Proaño L, Arellano-Treviño M A, Farrauto R J, Figueredo M, Jeong-Potter C, Cobo M. Mechanistic assessment of dual function materials, composed of Ru—Ni, Na₂O/Al₂O₃ and Pt—Ni, Na₂O/Al₂O₃, for CO₂ capture and methanation by in-situ DRIFTS. Appl Surf Sci 2020; 533:147469. https://doi.org/10.1016/j.apsusc.2020.147469.
[60] Chang K, Zhang H, Cheng M J, Lu Q. Application of Ceria in CO₂ Conversion Catalysis. ACS Catal 2020; 10:613-31. https://doi.org/10.1021/acscatal.9b03935.
[61] Le Sachê E, Pastor-Pérez L, Haycock B J, Villora-Picó J J, Sepúlveda-Escribano A, Reina T R. Switchable Catalysts for Chemical CO₂ Recycling: A Step Forward in the Methanation and Reverse Water-Gas Shift Reactions. ACS Sustain Chem Eng 2020; 8:4614-22. https://doi.org/10.1021/acssuschemeng.0c00551.

1. A method of converting carbon dioxide into a reaction product, the method comprising:
providing a switchable dual function material (DFM) loaded with carbon dioxide; and
contacting the switchable DFM loaded with carbon dioxide and a co-reactant, thereby causing the carbon dioxide to react with the co-reactant to produce the reaction product,
wherein the switchable DFM comprises an adsorbent, configured to adsorb carbon dioxide; and
a switchable catalyst configured to catalyse the conversion of carbon dioxide into a reaction product.

2. The method of claim 1, wherein providing the switchable DFM loaded with carbon dioxide comprises contacting a gas comprising carbon dioxide and the switchable DFM, optionally wherein the carbon dioxide is present in the gas at a concentration of less than 80 vol %.

3. (canceled)

4. The method of claim 1, wherein the method comprises selecting a specific co-reactant to provide a desired reaction product, wherein the co-reactant is selected from the group consisting of a hydrocarbon; hydrogen; water and oxygen. The hydrocarbon may be as defined above.

5. The method of claim 1, wherein the method comprises contacting the switchable DFM loaded with carbon dioxide and the co-reactant at a temperature between 0 and 1,500° C.

6. A switchable dual function material (DFM) comprising:
an adsorbent, configured to adsorb carbon dioxide; and
a switchable catalyst configured to catalyse the conversion of carbon dioxide into a reaction product.

7. The method of claim 1, wherein the switchable DFM comprises a support, and the adsorbent and the switchable catalyst are both disposed on the support.

8. The method of claim 7, wherein the support comprises or consists of a metal, a metal oxide, silica, a metal organic framework, a zeolite or a structured carbon, preferably wherein the support comprises or consists of cerium, aluminium, zirconium, titanium, silicon and/or an oxide thereof.

9. The method of claim 1, wherein the switchable catalyst is configured to catalyse two or more different chemical reactions selected from the group consisting of a dry reforming reaction; a CO₂ methanation; an RWGS reaction; a bi-reforming reaction; a tri-reforming reaction; a dehydrogenation reaction; and a hydrogenation reaction.

10. The method of claim 1, wherein the switchable catalysts comprise one or more metals and/or a metal phosphide, preferably wherein the one or more metals is or comprises one or more transition metals and the metal phosphide is a transition metal phosphide, optionally wherein the switchable catalyst may comprise one or more of nickel (Ni), ruthenium (Ru), cerium (Ce), zirconium (Zr), iron (Fe), and/or a phosphide thereof.

11. (canceled)

12. The method of claim 10, wherein the catalyst comprises nickel, optionally wherein the switchable catalyst comprises nickel and one or more additional components or promoters, and the or each additional component or promoter is ruthenium (Ru) or iron (Fe).

13. The method of claim 10, wherein the switchable catalyst comprises or consists of a nickel phosphide.

14. The method of claim 1, wherein the adsorbent is or comprises a metal, an oxide of a metal, a carbonate of a metal, a metal organic framework, a zeolite, silica and/or carbon, optionally wherein the adsorbent is or comprises an alkali metal, an alkaline earth metal and/or an oxide or carbonate thereof, preferably wherein the adsorbent is or comprises sodium oxide, potassium oxide and/or calcium oxide.

15. (canceled)

16. The method of claim 1, wherein the switchable DFM has a surface area of at least 100 m²/g and/or a pore volume of at least 0.2 cm³/g.

17. A method of producing a switchable DFM, the method comprising:
providing a support, wherein either the support is an adsorbent, and is configured to adsorb carbon dioxide, or the method comprises disposing an adsorbent on the support, wherein the adsorbent is configured to adsorb carbon dioxide; and
disposing a switchable catalyst on the support, wherein the switchable catalyst is configured to catalyse the conversion of carbon dioxide into a reaction product;
and thereby producing a switchable DFM.

18. The method of claim 17, wherein the method comprises disposing an adsorbent on the support, optionally wherein the method comprises disposing the adsorbent on the support prior to disposing the switchable catalyst on the support.

19. (canceled)

20. The method of claim 18, wherein disposing the adsorbent on the support comprises contacting the support with the adsorbent or an adsorbent precursor, optionally wherein contacting the support with the adsorbent or an adsorbent precursor comprises contacting the support with a solution or suspension comprising a solvent and the adsorbent or adsorbent precursor, to provide a further suspension and subsequently drying the further suspension to remove the solvent therefrom and provide a support with the adsorbent or adsorbent precursor disposed thereon.

21. (canceled)

22. The method of claim 20, wherein the method comprises contacting the support with an adsorbent precursor, and subsequently calcining the adsorbent precursor to provide the adsorbent.

23. The method of claim 17, wherein disposing the switchable catalyst on the support comprises contacting the support with the switchable catalyst or a switchable catalyst precursor, optionally wherein contacting the support with the switchable catalyst or switchable catalyst precursor comprises contacting the support with a solution or suspension comprising a solvent and the switchable catalyst or switchable catalyst precursor, to thereby provide a further suspension and subsequently drying the further suspension to remove the solvent therefrom and provide a support with the switchable catalyst or switchable catalyst precursor disposed thereon.

24. (canceled)

25. The method of claim 23, wherein the method comprises contacting the support with a switchable catalyst precursor, and subsequently calcining the switchable catalyst precursor to provide the switchable catalyst.

26. (canceled)

27. A method of capturing carbon dioxide, the method comprising contacting a gas comprising carbon dioxide and the switchable DFM defined by claim 6.
